(19) United States
(12) Patent Application Publication
(10) Pub. No.: US 2024/0092873 A1
(43) Pub. Date: Mar. 21, 2024

(54) ANTI-VIRAL THERAPEUTIC

(71) Applicant: University College Cardiff Consultants Limited, Cardiff South Glamorgan (GB)

(72) Inventors: Richard Stanton, Cardiff (GB); Edward Chung Yern Wang, Cardiff (GB)

(73) Assignee: University College Cardiff Consultants Limited, Cardiff South Glamorgan (GB)

(21) Appl. No.: 18/263,055

(22) PCT Filed: Jan. 27, 2022
(86) PCT No.: PCT/GB2022/050217

§ 371 Date: Jul. 26, 2023

(30) Foreign Application Priority Data
Jan. 27, 2021 (GB) 2101066.5

(51) Int. Cl. C07K 16/08; A61P 31/22

(52) U.S. Cl. CPC C07K 16/089; A61P 31/22; C07K 2317/41; C07K 2317/52; C07K 2317/56; C07K 2317/565; C07K 2317/622; C07K 2317/72; C07K 2317/732; C07K 2317/94

(57) ABSTRACT

The invention relates to an anti-viral composition comprising at least one, and ideally a plurality of, monoclonal antibodies, or fragments thereof; an immunogenic agent, vaccine or pharmaceutical composition comprising the afore anti-viral composition; said anti-viral composition, immunogenic agent, vaccine or said pharmaceutical composition for use in the treatment of or prevention of a viral infection; use of said anti-viral composition in the manufacture of a medicament to treat or prevent a viral infection; a combination therapeutic for use in the treatment or prevention of a viral infection comprising said anti-viral composition, immunogenic agent, vaccine or pharmaceutical composition in combination with at least one other therapeutic agent; and a method of treating or preventing a viral infection comprising administering said anti-viral composition, immunogenic agent, vaccine or said pharmaceutical composition to an individual having, or suspected of having, a viral infection.

### CROSS REFERENCE TO RELATED APPLICATIONS

This is the U.S. National Stage of International Application No. PCT/GB2022/050217, filed Jan. 27, 2022, which was published in English under PCT Article 21(2), which in turn claims the benefit of GB 2101066.5 filed Jan. 27, 2021. The PCT application is incorporated herein in its entirety.

### FIELD OF THE INVENTION

The invention relates to an anti-viral composition comprising at least one, and ideally a plurality of, monoclonal antibodies, or fragments thereof; an immunogenic agent, vaccine or pharmaceutical composition comprising the afore anti-viral composition; said anti-viral composition, immunogenic agent, vaccine or said pharmaceutical composition for use in the treatment of or prevention of a viral infection; use of said anti-viral composition in the manufacture of a medicament to treat or prevent a viral infection; a combination therapeutic for use in the treatment or prevention of a viral infection comprising said anti-viral composition, immunogenic agent, vaccine or pharmaceutical composition in combination with at least one other therapeutic agent; and a method of treating or preventing a viral infection comprising administering said anti-viral composition, immunogenic agent, vaccine or said pharmaceutical composition to an individual having, or suspected of having, a viral infection.

### INCORPORATION OF ELECTRONIC SEQUENCE LISTING

The electronic sequence listing, submitted herewith as a txt file named Sequence Listing_st25.txt (41480 bytes), created on Jul. 19, 2023, is herein incorporated by reference in its entirety.

### BACKGROUND OF THE INVENTION

Human Cytomegalovirus (HCMV, also known as human herpesvirus-5) is a nearly ubiquitous herpes virus that will infect over 90% of the population at some point in their life. Following primary infection, HCMV typically establishes a persistent infection that is kept under control by a healthy immune system. HCMV employs a multitude of immune-modulatory strategies to evade the host immune response. Examples of such strategies include inhibition of interferon (IFN) and IFN-stimulated genes, degradation of HLA to prevent antigen presentation to cytotoxic T cells and modulation of activating and inhibitory ligands to prevent natural killer (NK) cell function.

Though HCMV infection typically goes unnoticed in healthy individuals, reactivation from viral latency in immunocompromised individuals (e.g., HIV-infected persons, organ transplant recipients), acquisition of primary infection or infection with an additional strain in such individuals (e.g., during transplantation), can lead to serious disease. For example, HCMV is one of the major causes of graft failure and mortality in transplant recipients who require prolonged immunosuppression. Similarly, primary infection, reactivation, or acquisition of a secondary strain, during pregnancy can lead to transmission to the foetus, causing congenital abnormalities (e.g. blindness, deafness, intellectual disability). HCMV infection has also been linked with certain cancers.

Human cytomegalovirus (HCMV) therefore establishes lifelong infection in the face of robust humoral and cell-mediated immune responses and is a significant cause of morbidity and mortality in immunocompromised and immunonaive individuals.

HCMV infection in immunocompromised individuals is currently treated using purified plasma immunoglobulin (CMV-IGIV) and antiviral drugs, such as Ganciclovir (Cytovene) and Valganciclovir (Valcyte). Because CMV-IVIG is derived from donated human plasma, it is difficult to produce in large quantity, is undefined, and its use carries the risk of the transmission of infectious disease. Moreover, many drugs show significant toxicity, and drug-resistant HCMV strains have become increasingly common, often rendering current therapies ineffective.

A vaccine against HCMV is considered to be of the highest priority, particularly for the prevention of congenital disease, but one is not currently available. As a virus that persists lifelong, HCMV poses major challenges: it avoids being cleared by the immune response; it has evolved an exceptionally broad range of techniques to limit immune-activation; and so it poses a particular challenge for the development of methods to activate anti-viral immunity. Neutralising monoclonal antibodies (mAbs) therapies against HCMV have had only modest effects and/or have failed to meet primary endpoints in clinical trials, namely a reduction in viremia and/or the need for pre-emptive therapy.

One potential explanation for this lack of clinical efficacy lies in the biology of virus dissemination. Whilst spread of HCMV between individuals involves cell-free virus, which can be efficiently inhibited by neutralising antibodies, dissemination within a host relies primarily on direct cell-to-cell spread, which is resistant to neutralising antibodies, irrespective of the antibody repertoire of the donor. Thus, while classical monoclonal neutralising antibodies may have a role in preventing transmission between people, they are less effective at preventing the spread of virus within an individual.

We have therefore sought to prioritise antibody-based immunotherapeutic approaches that could target infected cells directly.

Natural Killer (NK) cells are crucial for virus control in vivo. This fact is highlighted by the impressive arsenal of HCMV-encoded immune-evasins that act in consort to suppress NK cell activation through the manipulation of ligands for activating inhibitory NK cell receptors. However, as well as working through these receptors, NK cells participate in antibody-dependent cellular cytotoxicity (ADCC). ADCC involves the activation of NK cells upon engagement of Fc receptors on the NK cell surface when the Fc portion of an antibody is bound to a target cell. In vivo, HCMV infection is associated with a dramatic expansion of ‘adaptive’ NK cells marked by the expression of CD94/NKG2C, CD57, and by the loss of FcεR1γ. These cells are exceptionally efficient at mediating ADCC and have been associated with protection from disease. Accordingly, ADCC may be an important mechanism of immune control during natural infection. In ADCC, antibodies act as critical stimulators of cellular immunity, rather than acting through virus neutralisation.

We have therefore investigated how ADCC operates in the context of an HCMV infection, and whether it can be exploited for therapeutic use. We have found that anti-HCMV antibodies can activate NK cells early after HCMV infection, prior to the production of new virions, and we have shown these antibodies have a remarkable capacity to overwhelm the potent HCMV-encoded NK cell evasion mechanisms in vitro.

Historically, we have used proteomics to characterise, in unparalleled detail, viral and host gene expression during HCMV infection, so revealing the ways by which the virus manipulates the host-cell to promote its survival, and to identify ways of counteracting the virus through antiviral restriction factors. We have now combined this technique with functional immunological screening to identify the targets on the infected cell surface that mediate anti-viral ADCC. Surprisingly, these techniques have revealed that the optimal ADCC targets are not the structural glycoproteins that are traditionally assumed to be ADCC targets, but immune-evasins that are expressed earlier during the viral lifecycle. The identification of these targets has enabled us to isolate human mAbs directed against them that can activate NK cells in response to HCMV infected cells.

Thus, we have identified optimal antigenic targets for the development of anti-viral therapeutics and so produced the first human mAbs, targeting only a single HCMV protein, that are sufficient to mediate enhanced virus control through ADCC; this is despite the presence of viral encoded immune-evasins.

### STATEMENTS OF INVENTION

According to a first aspect of the invention there is provided an anti-viral composition comprising at least one, and ideally a plurality of, monoclonal antibodies, or fragments thereof, having a plurality of different variable regions selected from the group comprising or consisting of:

a)
(SEQ ID NO: 1 D3)
DIQMTQSPSTLSASVGDRVTITCRASQSISSWLAWYQQKPGKAPKLLIYM
ASSLESGVPSRFSGSGSGTEFTLTISSLQPDDFATYYCQQYNSYPRTFGQ
GTKVEIK;
b)
(SEQ ID NO: 2 G3)
QSALTQPASVSGSPGQSITISCTGTSNDVGAYNSVSWYQQHPGKAPKLMI
YDVDNRPSGVSTRFSGSKSGNTASLTISGLQPDDEADYYCSSYTSRRTLG
VFGGGTKVTVL;
c)
(SEQ ID NO: 3 G4)
EIVLTQSPATLSLSPGERATLSCRASQSASSYVAWYQQKPGQAPRLLIYD
VSIRANGIPARFSGSGSGTDFALTISSLEPEDFALYYCQHRNNWGSTFGQ
GTRLEIK;
d)
(SEQ ID NO: 4 G11)
DIQMTQSPSTLSASVGDRVTITCRASQSISKWVAWYQLKSGKVPKLLIYQ
ASDLQSGVPTRFSGSGSGTEFTLTIRGLQSDDFATYYCQQFDHSPWTFGQ
GTKVEIK;
e)
(SEQ ID NO: 5 B2)
DIQMTQSPSTLSASVGDRVTITCRASQSVSGWLAWYQQKPGKAPKLLIYM
ASSLEGGVPSRFSGSGSGTEFTLTISSLQPDDFATYYCQQYNSYPRTFGQ
GTKVEIK;
f)
(SEQ ID NO: 6 C3)
QSVLTQPPSVSAAPGQKVTISCSGSSSNIGNNYVSWYQQLPGTAPKLLIY
DNNKRPSGIPDRFSGSKSGTSATLGITGLQTGDEADYYCGTWDSSLLEVV
FGGGTKLTVL;
g)
(SEQ ID NO: 7 E5)
QSVLTQPPSASGTPGQRVTISCSGGSSNIGSNPVNWYQQIPGTAPKLLIY
SDDQRPSGVPDRFSGSKSGSSASLAIRGLQSEDEADYFCAARDDSLNGPI
FGGGTKLTVL;
h)
(SEQ ID NO: 8 G2)
QSALTQPASVSGSPGQSITISCIGTSSDVGKNNLVSWYQQYPDKAPKLMI
YDVTKRPSGVSNRFSGSKSGNMASLTISGLQTEDEAHYYCCSYAGVGGHI
LWVFGGGTKVTVL;

and
i) a variable region that shares at least 85% identity with any one of variable regions a)-h) (SEQ ID NOs: 1-8).

In a preferred embodiment of the invention said monoclonal antibody/antibodies, or fragments thereof, have a plurality of light or heavy chain variable regions selected from the group comprising or consisting of sequences a)-i), including any combination thereof, most ideally light chain variable regions.

Yet more preferably, said monoclonal antibody/antibodies, or fragments thereof, have a plurality of light or heavy chain variable regions selected from the group comprising or consisting of: sequences a)-e), g) and i), including any combination thereof, most ideally light chains. In this embodiment of the invention, said anti-viral composition comprises a plurality of different variable regions comprising or consisting of a plurality of different sequences including any one or more of a)-e), g) and i), including any combination thereof such as any of the 6, 5, 4, 3, or 2 variable regions selected from the group comprising or consisting of a)-e), g) and i). In this embodiment, said variable regions ideally form part of one or more light chains of said antibody/antibodies/fragments.

In a further preferred embodiment of the invention said ant-viral is an anti-HMCV composition which is, ideally, therapeutically effective against HCMV infection.

Additionally, or alternatively, in a further aspect of the invention or a preferred embodiment of the invention, said anti-viral composition comprising at least one, and ideally a plurality of, monoclonal antibodies, or fragments thereof, having a plurality of different variable regions selected from the group comprising or consisting of:

j)
(SEQ ID NO: 9 D3)
EVQLVESGGDLVQPGGSLRLSCAASGFIVSSNYMSWVRQAPGKGLEWVS
VIHSDGPTFYADSVKGRFTISRDSSKNMLYLQMNSLRAEDTAVYYCTRG
EFASGLYGSAGSNAFDFWGQGTLVTVSS;
k)
(SEQ ID NO: 10 G3)
EVQLVESGGGLVQPGGSLRLSCVASTFTISPYWMSWVRQAPGKGLEWVA
NIKDDGSERYYVDSVKGRFTISRDNAKNSVFLQMNSLRAEDTATYYCAR
PGPDAFSTGWSNWFDPWGQGMLVTVSS;
l)
(SEQ ID NO: 11 G4)
QVQLQESGPGLVRPSQTLSLTCTVSGASITSGSYYWTWIRQPAGEGLEW
LGRINTRGNINYKPSLRSRLTFSVDTSKNQFSLQLSSVTAADSAVYFCA
RVGLYDTYYYFMDVWGKGTTVTVSS;
m)
(SEQ ID NO: 12 G11)
QVQLQESGPGLVRPSETLSLTCTVSGASVSAYYWTWIRHSPGRGLEWIG
DIYFNGKFNYNPSLESRVTISRGPSKTQLSLKLSSVTAADSAVYYCARI
GDSTMAPLYYFYYIDVWGKGTTVTVSS;
n)
(SEQ ID NO: 13 B2)
EVQLVESGGGLVQPGGSLRLSCAASAFTVSSMYMNWVRQAPGKGLEWV
SVIYSDGTTYYRDSVKGRFTISRDNSKNKVYLQMNSLRAEDTAVYYCAR
GEFASGWYGSAGSNAFDIWGRGTMVTVSS;
o)
(SEQ ID NO: 14 C3)
EVQLVQSGAEVKKPGASVKVSCKASGYTFTNYAISWVRQAPGQGLEWMG
WISAYNGNTNYAQKLQGRVTMTTDTSTSTAYMELRSLRSDDTAVYYCAR
VGTMVRGVIYNKRPYYYYYMDVWGKGTTVTVSS;
p)
(SEQ ID NO: 15 E5)
EVQLVQSGAEVRKPGSSVKLSCKASGGTFRNYAMSWMRQAPGQGFEWV
GGIVPFLGKTNYAQKFQGRVTISTDESTSTAYMELSRLTSDDTAVYFCA
RGPPPVMVRGIHRTGGDWFDPWGQGTLVTVSS;
q)
(SEQ ID NO: 16 G2)
EVQLVQSGAELKKPGSSVKVSCKASGGTFSFHAINWVRQAPGQGLEWMG
GIIPVSDTTNYAQKFHSRLTITADESTSTSYMQLTSLTDEDTAVYYCAR
EYGPVATGFDPWGQGTLVTVSS;

and
r) a variable region that shares at least 85% identity with any one of variable regions j)-q) (SEQ ID NO: 9-16).

More preferably, said variable region has at least 86, 87, 88, 89, 90, 91, 92, 93, 94, 95, 96, 97, 98 or 99% identity with any one of the variable regions a)-h) and/or j)-q) (SEQ ID NOs: 1-8 and 9-16).

In a preferred embodiment of the invention said monoclonal antibody/antibodies, or fragments thereof, have a plurality of heavy or light chain variable regions selected from the group comprising or consisting of sequences j)-r), including any combination thereof, most ideally heavy chains.

Yet more preferably, said monoclonal antibody/antibodies, or fragments thereof, have a plurality of heavy or light chain variable regions selected from the group comprising or consisting of sequences j)-n), p) and r), including any combination thereof, most ideally heavy chains. In this embodiment of the invention, said composition comprises a plurality of different variable regions comprising or consisting of a plurality of different sequences including any one or more of j)-n), p) and r) including any combination thereof such as any of the 6, 5, 4, 3, or 2 variable regions belonging to the group comprising or consisting of j)-n), p) and r). In this embodiment, said variable regions ideally form part of one or more heavy chains of said antibody/antibodies/fragments.

In a preferred embodiment of the invention said monoclonal antibody/antibodies, or fragments thereof, have a plurality of heavy and/or light chain variable regions selected from the group comprising or consisting of sequences a)-r), including any combination thereof, most ideally light chains selected from the group comprising a)-i) and heavy chains selected from the group comprising j)-r), including any combination thereof, most ideally light chains selected from the group comprising a)-e), g) and i) and heavy chains selected from the group comprising j)-n), p) and r), including any combination of thereof.

In yet a further preferred embodiment, said monoclonal antibody/antibodies, or fragments thereof, comprise at least one of the following combinations of heavy and/or light chain variable regions:
i) variable regions a) and j) (SEQ ID NO.s 1 & 9, D3);
ii) variable regions b) and k) (SEQ ID NO.s 2 & 10, G3);
iii) variable regions c) and l) (SEQ ID NO.s 3 & 11, G4);
iv) variable regions d) and m) (SEQ ID NO.s 4 & 12, G11);
v) variable regions e) and n) (SEQ ID NO.s 5 & 13, B2);
vi) variable regions f) and o) (SEQ ID NO.s 6 & 14, C3);
vii) variable regions g) and p) (SEQ ID NO.s 7 & 15, E5);
viii) variable regions h) and q) (SEQ ID NO.s 8 & 16, G2); and/or
ix) two variable regions, each one having at least 85% identity with one variable region selected from the group comprising a)-h) and one variable region selected from the group comprising j)-q).

More ideally, said variable region of part ix) has at least 86, 87, 88, 89, 90, 91, 92, 93, 94, 95, 96, 97, 98 or 99% identity with any one of the variable regions a)-h) and j)-q) (SEQ ID NOs: 1-8 and 9-16).

In a preferred embodiment at least two of the said pairs i)-ix) are used in the composition of the invention, yet more preferably still 3 or 4 pairs are used and whilst 5 or more pairs may used, maximum activity is achieved when using 4 pairs.

In a further preferred embodiment of the invention said anti-viral composition is an anti-HMCV composition which is, ideally, therapeutically effective against a HCMV infection and preventative against HCMV disease.

Human cytomegalovirus (HCMV), also called Human betaherpesvirus 5 or CMV, is a virus of the genus Cytomegalovirus, which in turn is a member of the viral family known as Herpesviridae or herpesviruses. Within Herpesviridae, HCMV belongs to the Betaherpesvirinae subfamily, which also includes cytomegaloviruses from other mammals.

Advantageously, in the context of ADCC, HCMV has a slow replication cycle, with virions not being produced in significant numbers until 72 h post infection, this observation presents a therapeutic opportunity enabling us to limit the dissemination of HCMV using our anti-viral agent.

In yet a further preferred embodiment of the invention said antibody/antibodies, or fragment(s) thereof, include(s) an Fc region and so, ideally, said antibody/antibodies or fragments(s) thereof is/are heavy chain(s), or a fragment(s) thereof, including both a variable region and an Fc region. As will be appreciated, said Fc region can be an alpha, mu, gamma, epsilon, or delta isotype Fc region, or a fusion protein thereof, more preferably a gamma isotype Fc region, and most preferably a gamma isotype Fc region, subclass 1 (IgG1).

In a further preferred embodiment, said Fc region comprises at least one Fc modification, such as but not limited to, an Fc modification to increase effector cell binding/function and/or increase serum half-life. As will be known to those skilled in the art, there are a number of known modifications to the Fc region which increase effector cell function through enhancement of the antibody's ability to mediate cellular cytotoxicity functions such as antibody dependent cell mediated cytotoxicity (ADCC) including, but not limited to, point mutations and/or glycosylation. Typically, these modifications increase the affinity of the Fc domain for the Fc Receptor (FcR) on cells.

In a preferred embodiment said Fc region, ideally, is a gamma type Fc region, or a fusion protein thereof, and it comprises one or point mutations in the Fc region, ideally, at one or more of the following amino acid positions: 234, 236, 239, 243, 292, 298, 300, 305, 330, 332, 333, 334, 396, including any combination of the afore mutations. (The afore point mutations are numbered having regard to the sequence structure of the known IgG Fc region fragment structure as is known to one skilled in the art and reviewed in Saunders K O (2019) Conceptual Approaches to Modulating Antibody Effector Functions and Circulation Half-Life. Front. Immunol. 10:1296. doi: 10.3389/fimmu.2019.01296. This belongs to the recognised EU numbering system of Kabat E. A., Wu T. T., Perry H. M., Gottesman K. S., Foeller C. (1991) Sequences of Proteins of Immunological Interest (U.S. Dept. of Health and Hum. Serv, Bethesda)). More preferably, said one or more point mutation(s) is/are selected from the group comprising: S298A, E333A, K334A, S239D, A330L, I332E, G236A, L234Y, G236W, F243L, R292P, Y300L, V305I, P396L, or a combination thereof. Most preferably, said point mutation(s) comprise(s) S239D and/or I332E modifications introduced into the Fc region of the Mab, or a fragment thereof. In the context of our technology, these Fc region modifications enhance binding to CD16 on NK cells. This has the effect of optimising the ability of our mAbs to activate ADCC.

Alternatively, or additionally, FcR binding can be increased by glycol-engineering of the Fc region. As is known to those skilled in the art, FcRs interact with the carbohydrates on the CH2 domain of the mAb Fc region and the composition of these carbohydrates/glycans has a substantial influence on effector function activity. Specifically, when the oligosaccharides in the Fc region of the antibody do not have any fucose sugar units (afucosylation), ADCC is increased. Therefore, in a further preferred embodiment, said Fc region modification comprises modifications to the glycosylation status of the Fc region, most preferably, afucosylation of the Fc region thereby providing for antibodies comprising an afucosylated Fc region. Afucosylation of the Fc region can be achieved by numerous means known to those skilled in the art such as, but not limited to, use of antibody secreting cells engineered to overexpress Beta-1,4-mannosyl-glycoprotein 4-beta-N-acetylglucosaminyltransferase or knock out of one or more FUT genes, including FUT8, which encodes for Alpha-(1,6)-fucosyltransferase required for fucosylation.

Yet further still, alternatively or additionally, said Fc region comprises an Fc modification to increase serum half-life. Therefore, in a further preferred embodiment, said Fc region is, ideally a gamma type Fc region or a fusion product thereof, and it comprises one or point mutations at amino acid position 250, 252, 254, 256 or 428, including any combination of the afore mutations. (The afore point mutations are numbered having regard to the sequence structure of the known IgG Fc region fragment structure as is known to one skilled in the art and reviewed in Saunders K O (2019) Conceptual Approaches to Modulating Antibody Effector Functions and Circulation Half-Life. Front. Immunol. 10:1296. doi: 10.3389/fimmu.2019.01296. This belongs to the recognised EU numbering system of Kabat E. A., Wu T. T., Perry H. M., Gottesman K. S., Foeller C. (1991) Sequences of Proteins of Immunological Interest (U.S. Dept. of Health and Hum. Serv, Bethesda). More preferably, said point mutation is selected from the group comprising: T250Q, M428L, M252Y, S254T, T256E, or any combination thereof.

In yet a further preferred embodiment of the invention said monoclonal antibody/antibodies, or their fragments, have different variable regions that bind specifically to a single protein, ideally said protein is UL141 and ideally, UL141 of HCMV (Uniprot accession number Q6RJQ3), including variant strains thereof, including UL141 the sequence having amino acid sequence SEQ ID NO: 29.

UL141 is a type I transmembrane glycoprotein and is known to have a potent Natural Killer cell evasion function. The UL141 viral sequence is well-conserved among clinical HCMV isolates, suggesting that antibodies targeting them could control a broad range of HCMV strains.

Reference herein to an antibody, or fragment thereof, refers to at least the part of the antibody that binds antigen and so includes at least a variable region, but in any case, the single antibody or the plurality of antibodies, or their fragments, include a plurality of different variable regions. Accordingly, the anti-viral composition of the invention may include a single one or type of antibody that includes a plurality of different variable regions, the number being determined by the type of antibody for example an IgG antibody can have up to four variable regions whereas an IgM antibody can have up to 20 variable regions, or the anti-viral composition of the invention may include a plurality of antibodies, or their fragments, each antibody or fragment including at least one variable region or a number of different variable regions whereby, in any case, said composition comprises or consists of a plurality of different variable regions and, ideally, 2 different variable regions, more ideally still, 3 different variable regions and most preferably at least 4, 5, 6, 7, 8, 9, 10, 11, 12, 13, 14, 15, 16 different variable regions.

In a preferred embodiment of the invention each different variable region targets a different part, or epitope, of the target antigen, ideally UL141.

Ideally said antibody fragment comprises at least one variable region and at least one Complementarity Determining Region (CDR) thereof. More ideally still, said antibody fragment comprises a plurality of different variable regions and a plurality of Complementarity Determining Regions (CDRs). Yet more preferably still, said antibody fragment also includes an Fc region and ideally a modified Fc region as herein described.

In a preferred embodiment of the invention said antibody or antibodies, or fragments thereof, comprise at least 2 different variable regions.

Reference herein to the term therapeutically active is reference to an anti-viral that can activate an immune response, such as ADCC, against virally infected cells typically, but not exclusively, by activating NK cells. More specifically, reference herein to the term therapeutically active is reference to an anti-viral that can activate an immune response, such as ADCC, against cells infected with virus, preferably HCMV.

In either event, immune activation results in killing of virally infected cells and so, ultimately to viral clearance. Most advantageously this immune effect is not restricted by cell type and so the anti-viral agent of the invention is likely to be effective throughout an organism/individual.

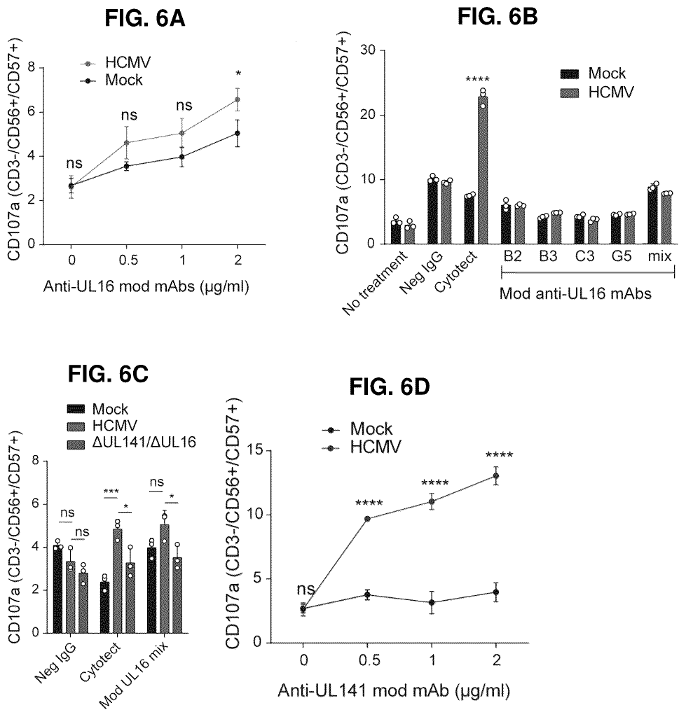

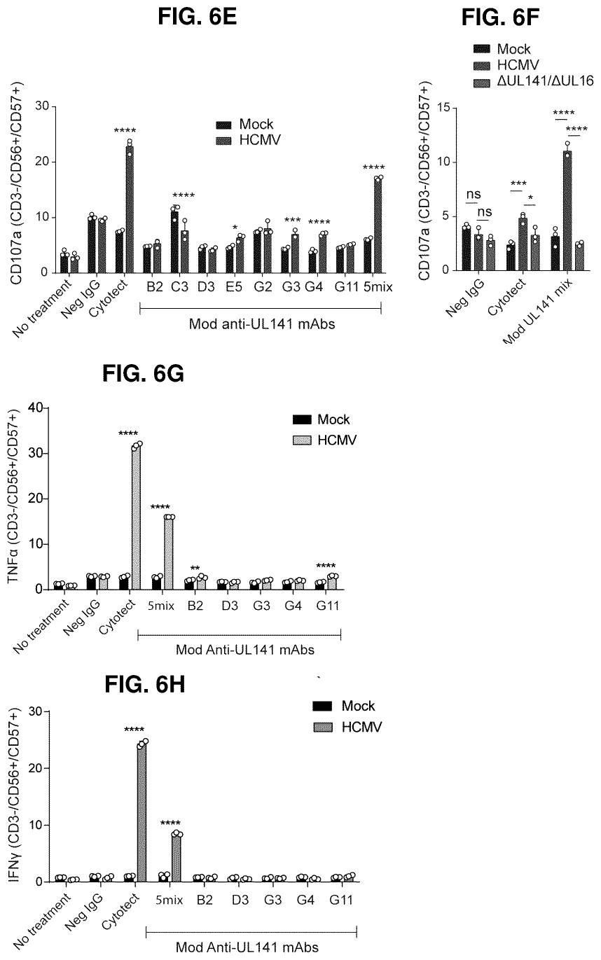

Without wishing to be bound by theory, we consider ADCC is efficiently achieved against HCMV using a plurality of the claimed different variable regions in a single or a plurality of anti-UL141 antibodies. Whilst, individually, our mAbs activated ADCC, a combination of variable regions or antibodies including same was successful at activating ADCC, almost as effectively as a reference activation (Cytotect), despite being used at a 40-fold lower concentration (FIG. 6D-E). Moreover, this activation was highly specific, because it was not apparent when a virus lacking the cognate antigen was used (FIG. 6F). Furthermore, these different variable regions or antibodies were also capable of activating NK cells, indicating potent antiviral effector functions.

We have therefore identified a viral-derived, cell-surface target (UL141) for the development of a novel anti-viral immunotherapy or vaccination strategy. With this knowledge we have generated what we believe to be the first human anti-viral employing antibody/antibodies targeting a single viral antigen that is/are sufficient to activate ADCC, especially in the context of HCMV infection.

According to a further aspect of the invention there is provided an immunogenic agent or vaccine comprising the anti-viral composition as disclosed herein together with a pharmaceutically acceptable excipient or carrier.

According to a further aspect of the invention there is provided a pharmaceutical composition comprising the anti-viral composition as disclosed herein together with a pharmaceutically acceptable excipient or carrier.

Most suitably said immunogenic agent or said vaccine or said pharmaceutical composition is formulated for human or veterinary use.

Suitable pharmaceutical excipients are well known to those of skill in the art. Pharmaceutical compositions may be formulated for administration by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association the anti-viral composition as defined herein with the carrier. In general, the formulations are prepared by uniformly and intimately bringing into association the anti-viral composition with liquid carriers or finely divided solid carriers or both and then, if necessary, shaping the product. The invention extends to methods for preparing a pharmaceutical composition comprising bringing the anti-viral composition, as defined herein together in conjunction or association with a pharmaceutically or veterinary acceptable carrier or vehicle.

Parenteral formulations will generally be sterile.

For topical application to the skin, the composition may be made up into a cream, ointment, jelly, solution or suspension etc. Cream or ointment formulations that may be used for the drug are conventional formulations well known in the art, for example, as described in standard textbooks of pharmaceutics such as the British Pharmacopoeia.

The precise amount of a composition as defined herein which is therapeutically effective, and the route by which such compound is best administered, is readily determined by one of ordinary skill in the art. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for any other reasons. Other factors include the desired period of treatment. In the event that a response in a subject is insufficient at the initial doses applied, higher doses (or effectively higher doses by a different, more localized delivery route) may be employed to the extent that patient tolerance permits.

According to a further aspect of the invention there is provided a combination therapeutic comprising the above anti-viral composition in combination with at least one other therapeutic agent.

Ideally, said at least one other therapeutic agent is an agent used to treat a viral infection or its associated symptoms.

According to a further aspect of the invention there is provided said anti-viral composition or said pharmaceutical composition or said immunogenic agent or said vaccine or said combination therapeutic for use in the treatment, or prevention, of a viral infection, most preferably a HCMV infection.

According to a yet further aspect of the invention there is provided the use of said anti-viral composition or said pharmaceutical composition or said immunogenic agent or said combination therapeutic in the manufacture of a medicament to treat, prevent or vaccinate against a viral infection, most preferably a HCMV infection.

According to a further aspect of the invention there is provided a method of treating a viral infection comprising administering said anti-viral composition or said pharmaceutical composition or said immunogenic agent or said vaccine or said combination therapeutic to an individual having, or suspected of having, a viral infection.

In preferred method of the invention said anti-viral composition or said pharmaceutical composition or said immunogenic agent or said vaccine or said combination therapeutic is administered shortly after infection or likely infection or after exposure to said virus and is, ideally, prior to 72 h post infection. This presents a favourable therapeutic opportunity enabling one to limit the dissemination of HCMV using our anti-viral agent.

Most preferably said infection is a HCMV infection.

According to a further aspect of the invention there is provided a method of vaccinating against a viral infection comprising administering said anti-viral composition or said pharmaceutical composition or said immunogenic agent or said vaccine or said combination therapeutic to an individual.

Most preferably said infection is a HCMV infection.

In a preferred embodiment of this aspect of the invention, said individual is human, although the composition may also be used to treat animals, ideally mammals.

According to yet a further aspect of the invention there is provided a multi-specific antibody, or fragment thereof, having at least one variable region that binds UL141 and at least one variable region that binds CD16.

Reference herein to UL141 refers to UL141 of HCMV (Uniprot accession number Q6RJQ3), having the has the following amino acid sequence (SEQ ID NO: 29): In yet a further preferred embodiment, said, at least one UL141 variable region binds to SEQ ID NO: 29 or an amino acid sequence that is at least about 80% similar or identical to SEQ ID NO: 29. For example, the at least one UL141 variable region binds to an amino acid sequence that is at least about 82%, 83%, 84%, 85%, 86%, 87%, 88%, 89%, 90%, 91%, 92%, 93%, 94%, 95%, 96%, 97%, 98% or at least about 99% similar or identical to SEQ ID NO: 29. For example, the at least one UL141 variable region may bind to an amino acid sequence that is up to 100% identical to SEQ ID NO: 29.

Amino acid sequences with a degree of similarity or identity may be determined using the BLAST® (Basic Local Alignment Search Tool) provided by National Center for Biotechnology Information (NCBI).

In a preferred embodiment, the multi-specific antibody, or fragment thereof, retains specificity and affinity for their UL141 and CD16 antigens.

In a further preferred embodiment, said at least one UL141 variable region comprises a least one variable region selected from the group comprising or consisting of:

a)
(SEQ ID NO: 1 D3)
DIQMTQSPSTLSASVGDRVTITCRASQSISSWLAWYQQKPGKAPKLLIY
MASSLESGVPSRFSGSGSGTEFTLTISSLQPDDFATYYCQQYNSYPRTF
GQGTKVEIK;
b)
(SEQ ID NO: 2 G3)
QSALTQPASVSGSPGQSITISCTGTSNDVGAYNSVSWYQQHPGKAPKLM
IYDVDNRPSGVSTRFSGSKSGNTASLTISGLQPDDEADYYCSSYTSRRT
LGVFGGGTKVTVL;
c)
(SEQ ID NO: 3 G4)
EIVLTQSPATLSLSPGERATLSCRASQSASSYVAWYQQKPGQAPRLLIY
DVSIRANGIPARFSGSGSGTDFALTISSLEPEDFALYYCQHRNNWGSTF
GQGTRLEIK;
d)
(SEQ ID NO: 4 G11)
DIQMTQSPSTLSASVGDRVTITCRASQSISKWVAWYQLKSGKVPKLLIY
QASDLQSGVPTRFSGSGSGTEFTLTIRGLQSDDFATYYCQQFDHSPWTF
GQGTKVEIK;
e)
(SEQ ID NO: 5 B2)
DIQMTQSPSTLSASVGDRVTITCRASQSVSGWLAWYQQKPGKAPKLLIY
MASSLEGGVPSRFSGSGSGTEFTLTISSLQPDDFATYYCQQYNSYPRTF
GQGTKVEIK;
f)
(SEQ ID NO: 6 C3)
QSVLTQPPSVSAAPGQKVTISCSGSSSNIGNNYVSWYQQLPGTAPKLLI
YDNNKRPSGIPDRFSGSKSGTSATLGITGLQTGDEADYYCGTWDSSLLE
VVFGGGTKLTVL;
g)
(SEQ ID NO: 7 E5)
QSVLTQPPSASGTPGQRVTISCSGGSSNIGSNPVNWYQQIPGTAPKLLI
YSDDQRPSGVPDRFSGSKSGSSASLAIRGLQSEDEADYFCAARDDSLNG
PIFGGGTKLTVL;
h)
(SEQ ID NO: 8 G2)
QSALTQPASVSGSPGQSITISCIGTSSDVGKNNLVSWYQQYPDKAPKLM
IYDVTKRPSGVSNRFSGSKSGNMASLTISGLQTEDEAHYYCCSYAGVGG
HILWVFGGGTKVTVL;

i) a variable region that shares at least 85% identity with any one of variable regions a)-h (SEQ ID NOs: 1-8);

j)
(SEQ ID NO: 9 D3)
EVQLVESGGDLVQPGGSLRLSCAASGFIVSSNYMSWVRQAPGKGLEWVS
VIHSDGPTFYADSVKGRFTISRDSSKNMLYLQMNSLRAEDTAVYYCTRG
EFASGLYGSAGSNAFDFWGQGTLVTVSS;
k)
(SEQ ID NO: 10 G3)
EVQLVESGGGLVQPGGSLRLSCVASTFTISPYWMSWVRQAPGKGLEWVA
NIKDDGSERYYVDSVKGRFTISRDNAKNSVFLQMNSLRAEDTATYYCAR
PGPDAFSTGWSNWFDPWGQGMLVTVSS;
l)
(SEQ ID NO: 11 G4)
QVQLQESGPGLVRPSQTLSLTCTVSGASITSGSYYWTWIRQPAGEGLEW
LGRINTRGNINYKPSLRSRLTFSVDTSKNQFSLQLSSVTAADSAVYFCA
RVGLYDTYYYFMDVWGKGTTVTVSS;
m)
(SEQ ID NO: 12 G11)
QVQLQESGPGLVRPSETLSLTCTVSGASVSAYYWTWIRHSPGRGLEWIG
DIYFNGKFNYNPSLESRVTISRGPSKTQLSLKLSSVTAADSAVYYCARI
GDSTMAPLYYFYYIDVWGKGTTVTVSS;
n)
(SEQ ID NO: 13 B2)
EVQLVESGGGLVQPGGSLRLSCAASAFTVSSMYMNWVRQAPGKGLEWV
SVIYSDGTTYYRDSVKGRFTISRDNSKNKVYLQMNSLRAEDTAVYYCAR
GEFASGWYGSAGSNAFDIWGRGTMVTVSS;
o)
(SEQ ID NO: 14 C3)
EVQLVQSGAEVKKPGASVKVSCKASGYTFTNYAISWVRQAPGQGLEWMG
WISAYNGNTNYAQKLQGRVTMTTDTSTSTAYMELRSLRSDDTAVYYCAR
VGTMVRGVIYNKRPYYYYYMDVWGKGTTVTVSS;
p)
(SEQ ID NO: 15 E5)
EVQLVQSGAEVRKPGSSVKLSCKASGGTFRNYAMSWMRQAPGQGFEWV
GGIVPFLGKTNYAQKFQGRVTISTDESTSTAYMELSRLTSDDTAVYFCA
RGPPPVMVRGIHRTGGDWFDPWGQGTLVTVSS;
q)
(SEQ ID NO: 16 G2)
EVQLVQSGAELKKPGSSVKVSCKASGGTFSFHAINWVRQAPGQGLEWMG
GIIPVSDTTNYAQKFHSRLTITADESTSTSYMQLTSLTDEDTAVYYCAR
EYGPVATGFDPWGQGTLVTVSS;

and
r) a variable region that shares at least 85% identity with any one of variable regions j)-q)) (SEQ ID NOs: 9-16).

More preferably, said variable region of i) and r) has at least 86, 87, 88, 89, 90, 91, 92, 93, 94, 95, 96, 97, 98 or 99% identity with any one of variable regions a)-h) and j)-q) (SEQ ID NOs: 1-16).

Yet more preferably, said at least one UL141 variable region comprises a least one variable region selected from the group comprising or consisting of: a)-e), g) and i) and j)-n), p) and r), including any combination thereof.

In a preferred embodiment of the invention, said multi-specific antibody, or fragment thereof, has a plurality of heavy and/or light chain variable regions selected from the group comprising or consisting of sequences a)-r), including any combination thereof, most ideally light chains selected from the group comprising a)-h) and i) and heavy chains selected from the group comprising j)-q) and r), including any combination thereof, most ideally light chains selected from the group comprising a)-e), g) and i) and heavy chains selected from the group comprising j)-n), p) and r), including any combination of thereof.

In yet a further preferred embodiment, said at least one UL141 variable region comprises at least one of the following combinations of variable regions:
i) variable regions a) and j) (SEQ ID NO.s: 1 & 9 D3);
ii) variable regions b) and k) (SEQ ID NO.s: 2 & 10 G3);
iii) variable regions c) and l) (SEQ ID NO.s: 3 & 11 G4);
iv) variable regions d) and m) (SEQ ID NO.s: 4 & 12 G11);
v) variable regions e) and n) (SEQ ID NO.s: 5 & 13 B2);
vi) variable regions f) and o) (SEQ ID NO.s: 6 & 14 C3);
vii) variable regions g) and p) (SEQ ID NO.s: 7 & 15 E5);
viii) variable regions h) and q) (SEQ ID NO.s: 8 & 16 G2); and/or
ix) two variable regions, each one having at least 85% identity with one variable region selected from the group comprising a)-r).

More ideally, said variable region of part ix) has at least 86, 87, 88, 89, 90, 91, 92, 93, 94, 95, 96, 97, 98 or 99% identity with any one of the variable regions a)-r) (SEQ ID NOs: 1-16).

Reference herein to CD16, also known as FcγRIII, is reference to a molecule found on the surface of Natural Killer (NK) cells, neutrophils, monocytes, macrophages, and certain T cells. In humans, it exists in two different forms: FcγRIIIa (CD16a) and FcγRIIIb (CD16b), which have 96% sequence similarity in the extracellular immunoglobulin binding region, with CD16b only expressed on neutrophils. CD16 is an activating receptor, typically binding the Fc region of IgG antibodies, triggering the antibody-dependent cell-mediated cytotoxicity (ADCC) of NK cells. NK cells are cytotoxic, IFN-γ-producing innate lymphocytes that are considered to constitute the first line of defence against virus-infected cells and cancer cells. The cytotoxic potential of NK cells can be utilized by redirecting NK cell lysis to target cells and stimulating the activating receptor CD16A, expressed on the cell surface of NK cells. In the present case, through selective targeting of HCMV infected cells utilising the UL141 binding variable region(s) disclosed herein, along with targeting CD16, one can potentiate the NK cell response leading to effective cell killing to provide a potent anti-HCMV therapeutic.

Preferably, CD16 refers to human CD16a and having the following amino acid sequence (SEQ ID NO: 30):

MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLFGSKNVSSETVNITITQGLAV
STISSFFPPGYQVSFCLVMVLLFAVDTGLYFSVKTNIRSSTRDWKDHKF
KWRKDPQDK

Thus, in certain embodiments, the CD16 variable region binds to SEQ ID NO: 30 or an amino acid sequence that is at least about 80% similar or identical to SEQ ID NO: 30. For example, the CD16 variable region may bind to an amino acid sequence that is at least about 82%, 83%, 84%, 85%, 86%, 87%, 88%, 89%, 90%, 91%, 92%, 93%, 94%, 95%, 96%, 97%, 98% or at least about 99% similar or identical to SEQ ID NO: 30. For example, the antibodies or antibody fragments may bind to an amino acid sequence that is up to 100% identical to SEQ ID NO: 30.

In a further preferred embodiment, said at least one CD16 variable region comprises a least one variable region selected from the group comprising or consisting of:

a)
(SEQ ID NO: 31)
QVQLVESGGGLVQPGGSLRLSCAASGLTFSSYNMGWFRQAPGQGLEAVA
SITWSGRDTFYADSVKGRFTISRDNSKNTLYLQMNSLRAEDTAVYYCAA
NPWPVAAPRSGTYWGQGTLVTVSS;
b)
(SEQ ID NO: 32)
EVQLVESGGGVVRPGGSLRLSCAASGFTFDDYGMSWVRQAPGKGLEWV
SGINWNGGSTGYADSVKGRFTISRDNAKNSLYLQMNSLRAEDTAVYYCA
RGRSLLFDYWGQGTLVTVSRGGGGSGGGGSGGGGSSELTQDPAVSVALG
QTVRITCQGDSLRSYYASWYQQKPGQAPVLVIYGKNNRPSGIPDRFSGS
SSGNTASLTITGAQAEDEADYYCNSRDSSGNHVVFGGGTKLTVL;
c)
(SEQ ID NO: 33)
SYVLTQPSSVSVAPGQTATISCGGHNIGSKNVHWYQQRPGQSPVLVIYQ
DNKRPSGIPERFSGSNSGNTATLTISGTQAMDEADYYCQVWDNYSVLFG
GGTKLTVLGGSGGSGGSGGSGGSGGSGGSQVQLVQSGAEVKKPGESLKV
SCKASGYTFTSYYMHWVRQAPGQGLEWMGAIEPMYGSTSYAQKFQGRVT
MTRDTSTSTVYMELSSLRSEDTAVYYCARGSAYYYDFADYWGQGTLVTV
SS;

and/or
d) a variable region that shares at least 85% identity with any one of variable regions a)-c)

More ideally, said variable region of part d) has at least 86, 87, 88, 89, 90, 91, 92, 93, 94, 95, 96, 97, 98 or 99% identity with any one of the variable regions a)-c) (SEQ ID NOs: 31-33).

In a preferred embodiment of the invention, said multi-specific antibody, or fragment thereof, has a plurality of heavy and/or light chain variable regions selected from the group comprising or consisting of sequences a)-c) (SEQ ID Nos: 31-33), including any combination thereof.

In certain embodiments, the multi-specific antibody binds UL141 and CD16 that are cell surface expressed. As used herein, the expression “cell surface-expressed” means one or more UL141 and/or CD16 protein(s) that is/are expressed on the surface of a cell in vitro or in vivo, such that at least a portion of a UL141 and/or a CD16 protein is exposed on the extracellular side of the cell membrane to drive ADCC against HCMV infected cells.

In some examples, the antibody or binding fragment thereof disclosed herein is a recombinant antibody or fragment. Recombinant antibodies or fragments thereof are antibodies or fragments that have been produced using recombinant antibody coding genes or that comprise parts derived from two different species.

The term variable region used herein refers to only a part of the structure of an antibody but includes the at least one UL141 and at least one CD16 variable region (V_{L}/V_{H}) as disclosed herein.

The multi-specific antibody may, for example, comprise, or consist of one or more Fab region(s) (each made from one heavy chain and one light chain, with each contributing one constant domain and one variable domain). For example, the multi-specific antibody may comprise or consist of one Fab region or the multi-specific antibody may comprise or consist of two Fab regions (F(ab)2).

The multi-specific antibody may, for example, comprise or consist of one or more light chain variable regions and one or more heavy chain variable regions. The light chain variable region(s) and heavy chain variable region(s) may, for example, be part of the same polypeptide and may, for example, be joined by one or more flexible linker sequence(s). The multi-specific antibody may, for example, comprise or consist of more than one light chain variable region and/or more than one heavy chain variable region which may, for example, be part of the same polypeptide and may, for example, be joined by one or more flexible linker sequence(s). A multi-specific antibody fragment comprising or consisting of a light chain variable region and a heavy chain variable region or a single polypeptide may, for example, be referred to as a single-chain variable fragment (scFv). The scFvs may, for example, be engineered to non-covalently bind to other scFvs (which may be the same or different) to form bivalent molecules referred to as diabodies. The scFvs may, for example, comprise more than one light chain variable regions and/or more than one heavy chain variable region in the same polypeptide (e.g. more than one scFv on the same polypeptide) and be referred to as tandem scFvs.

The multi-specific antibody fragment may, for example, comprise or consist of a single heavy chain (including constant and variable domains) and a single light chain (including constant and variable domains). The constant domains of the heavy chain and the light chain may each independently be complete or truncated.

The multi-specific antibody may, for example, comprise or consist of two heavy chains (both including constant and variable domains) and two light chains (both including constant and variable domains) where the constant domains of one or more of the heavy and light chains are truncated.

The multi-specific antibody, or fragment thereof, may comprise one or more Fv polypeptides, for example, a single-chain Fv (scFv), tandem single-chain Fv ((scFv)2) consisting of two scFvs joined in a single polypeptide, diabody (db), single chain diabody (scDb), tandem diabody (TandAb®), minibody/mini-antibody, Fab, F(ab′)2 or dual affinity retargeting antibodies (DART™) As will be appreciated, said multi-specific antibody when binding ULL141 and CD16 is a bispecific antibody, optionally and ideally wherein said bispecific antibody or fragment thereof is capable of mediating antibody dependent cellular cytotoxicity.

Such an arrangement can be achieved in the absence of other typical antibody domains, such as constant domains, in which case said antibody is said to be a Bi-specific Enhanced Killer Engager (or BiKE), this antibody configuration is known to those skilled in the art.

In yet a further preferred embodiment still, the multi-specific antibody further comprises at least one cytokine domain containing at least one cytokine, that further increases NK cell activation through binding of the cognate IL receptor on NK cells, including but not limited to pegylated cytokines and Fc fusion cytokines, and modified versions thereof. Non-limiting examples of cytokines that can be used in connection with these embodiments include IL-15, IL-2, IL-12, IL-21, IL-17, IL-18, IL-23, IL-27, and IL-6 containing domains and modified versions thereof. Most preferably the cytokine domain comprises IL-15 and so use of the multi-specific antibody essentially involves administering IL-15. As will be appreciated, when including such a further additional cytokine domain, said multi-specific antibody is said to be a tri-specific Enhanced Killer Engager (or TriKE), these antibody configurations are known to those skilled in the art.

In a preferred embodiment, said at least one cytokine domain selected from the group comprising or consisting of:

a)
(IL15N72D SEQ ID NO: 34)
NWVNVISDLKKIEDLIQSMHIDATLYTESDVHPSCKVTAMKCFLLELQV
ISLESGDASIHDTVENLIILANDSLSSNGNVTESGCKECEELEEKNIKE
FLQSFVHIVQMFINTS;
b)
(IL15 SEQ ID NO: 35)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

and/or
c) or an amino acid sequence that is at least about 80% similar or identical to SEQ ID NO: 34-35.

More ideally, said amino acid of part b) has at least 81, 82, 83, 85, 85, 86, 87, 88, 89, 90, 91, 92, 93, 94, 95, 96, 97, 98 or 99% identity or similarity with a-b) (SEQ ID NO: 34-35).

In yet a further preferred embodiment, the multi-specific antibody further comprises at least one variable region that binds Serum Albumin (SA), more preferably human serum albumin (HSA). It has been found that serum half-life can be extended by fusion of the multi-specific antibody with a HSA binding variable region.

Preferably, HSA has the following amino acid sequence (SEQ ID NO: 36):

MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

Thus, in certain embodiments, the HSA variable region binds to SEQ ID NO: 36 or an amino acid sequence that is at least about 80% similar or identical to SEQ ID NO: 35. For example, the HSA variable region may bind to an amino acid sequence that is at least about 82%, 83%, 84%, 85%, 86%, 87%, 88%, 89%, 90%, 91%, 92%, 93%, 94%, 95%, 96%, 97%, 98% or at least about 99% similar or identical to SEQ ID NO: 35. For example, the antibodies or antibody fragments may bind to an amino acid sequence that is up to 100% identical to SEQ ID NO: 36.

In a preferred embodiment, the multi-specific antibody may comprise 2, 3, 4, 5 or 6 HSA variable regions.

In a further preferred embodiment, said at least one HSA variable region comprises a least one variable region selected from the group comprising or consisting of:

a)
(SEQ ID NO: 37)
EVQLLESGGGLVQPGGSLRLSCAVSGIDLSNYAINWVRQAPGKGLEWIG
IIWASGTTFYATWAKGRFTISRDNSKNTVYLQMNSLRAEDTAVYYCART
VPGYSTAPYFDLWGQGTLVTVSS;
b)
(SEQ ID NO: 38)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGGGTKVEIK;

and/or
c) a variable region that shares at least 85% identity with any one of variable regions a)-b)

More ideally, said variable region of part c) has at least 86, 87, 88, 89, 90, 91, 92, 93, 94, 95, 96, 97, 98 or 99% identity with any one of the variable regions a)-b) (SEQ ID NOs: 37-38).

In a preferred embodiment of the invention, said multi-specific antibody, or fragment thereof, has a plurality of heavy and/or light chain variable regions selected from the group comprising or consisting of sequences a)-b) (SEQ ID Nos: 37-38), including any combination thereof.

We have determined that the dual binding properties of the multi-specific antibody provides for superior UL141 binding that provides for enhanced ADCC against HCMV infected cells. Indeed, it was found that, for each type of multi-specific antibody in accordance with the invention that was tested, i.e., when including the UL141 and CD16 binding variable regions, superior NK cell activation was achieved compared with the use of an antibody specific only for UL141 binding, and so lacking the CD16 binding variable.

According to a further aspect of the invention there is provided one or more polynucleotides encoding the multi-specific antibody of the invention, or a fragment thereof, as disclosed herein.

According to a yet further aspect of the invention there is provided one or more vectors comprising the one or more polynucleotides as disclosed herein.

The vectors preferably include elements which allow expression of said polynucleotides in a selected host cell. The vectors are, for example plasmid or viral vectors, and are useful for transforming host cells in order to clone or express the said antibody or polynucleotides disclosed herein. The vectors usually include a promoter, signals for initiation and termination of translation, as well as appropriate regions of regulation of transcription. The vector may optionally possess signals specifying the secretion of the translated protein. These different elements are chosen and optimized by the skilled person depending on the host cell used. Such vectors are prepared by methods commonly used by those skilled in the art, and the resulting vectors can be introduced into an appropriate host by standard methods, such as lipofection, electroporation, heat shock, or chemical methods.

According to another aspect of the invention there is provided at least one host cell comprising the one or more polynucleotides as disclosed herein, or the one or more vectors as disclosed herein. The cell may, for example, be referred to as a recombinant and/or host cell. The cell may, for example, be an established cell line (a cell that demonstrates the potential for indefinite subculture in vitro).

The cell may, for example, be a hybridoma. The cell may, for example, be prokaryotic (e.g., Escherichia coli) or eukaryotic (e.g., protist cell, animal cell (e.g., mammalian cell such as CHO or COS or HEK 293 cells, avian cell, insect cell such as Sf9 cell), plant cell, fungal cell (e.g., yeast cell such as Saccharomyces cerevisiae)).

In accordance with a further aspect of the present invention there is provided a pharmaceutical composition comprising the multi-specific antibody, or fragment thereof, described herein and a pharmaceutically acceptable carrier, excipient, or diluent. Preferably, said pharmaceutical composition comprises a pharmaceutically effective amount of said multi-specific antibody, or fragment thereof.

According to a further aspect of the invention there is provided a combination therapeutic comprising the multi-specific antibody, or fragment thereof, in combination with at least one other therapeutic agent.

Ideally, said at least one other therapeutic agent is an agent used to treat a viral infection or its associated symptoms.

According to a further aspect of the invention there is provided said multi-specific antibody, or fragment thereof, or said pharmaceutical composition or combination therapeutic for use in the treatment, or prevention, of a viral infection most preferably HCMV.

According to a yet further aspect of the invention there is provided the use of the multi-specific antibody, or fragment thereof, or said pharmaceutical composition or combination therapeutic in the manufacture of a medicament to treat, or prevent, a viral infection, most preferably HCMV.

According to a further aspect of the invention there is provided a method of treating or preventing a viral infection, most preferably HCMV, comprising administering said multi-specific antibody, or fragment thereof, or said pharmaceutical composition or combination therapeutic to an individual.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

In accordance with a further aspect of the present invention there is provided non-therapeutic (e.g., in vitro) use of the multi-specific antibody, or fragment thereof, of the present invention. For example, the antibody or binding fragment(s) thereof may be used for diagnosing, detecting or monitoring, a disease.

Preferred features of each aspect of the invention may be as described in connection with any of the other aspects.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

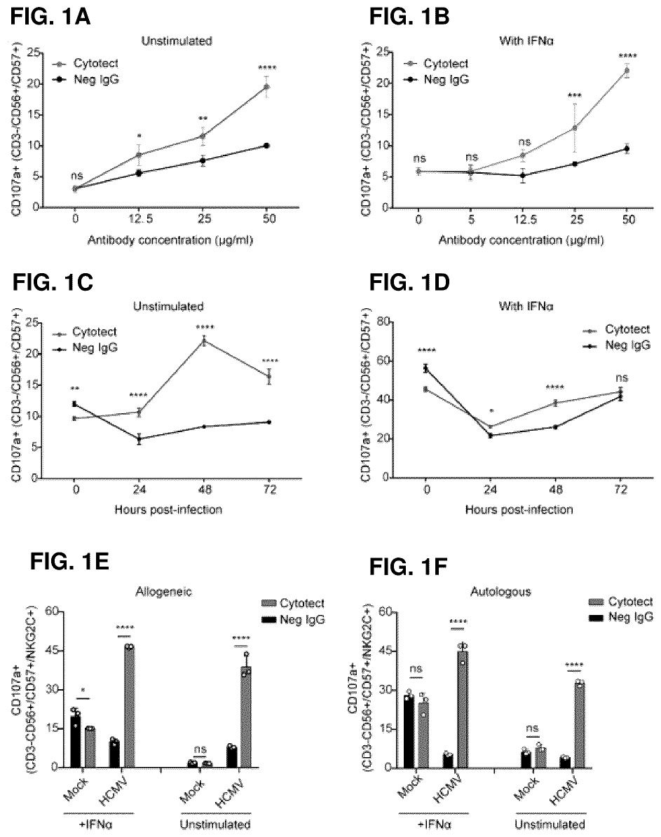

FIGS. 1A-1F: Characterisation of ADCC mediated NK cell activation against HCMV infected fibroblasts. Human fetal foreskin fibroblasts (HFFFs) immortalized with human telomerase reverse transcriptase (HF-TERTs) or similarly immortalized autologous skin fibroblasts (SFs) were infected with HCMV strain Merlin. Mock-infected HF-TERTs or SFs were included as controls. (A, B) Percent degranulation of CD56⁺ CD57⁺ NK cells among peripheral blood mononuclear cells (PBMCs) in the presence of HF-TERTs infected for 48 h with HCMV and different concentrations of either Cytotect or seronegative IgGs. PBMCs were either untreated (A) or pretreated for 18 h with IFN-α (B). (C, D) Percent degranulation of CD56⁺ CD57⁺ NK cells among PBMCs in the presence of HF-TERTs infected for 24 h, 48 h, or 72 h with HCMV and either Cytotect or seronegative IgGs (each at 50 μg/ml). PBMCs were either untreated (C) or pretreated for 18 h with IFN-α (D). (E, F) Percent degranulation of CD56⁺ CD57⁺ NKG2C⁺ NK cells among PBMCs in the presence of HF-TERTs (E) or SFs (F) infected for 48 h with HCMV and either Cytotect or seronegative IgGs (each at 50 μg/ml). Experiments are representative of at least three experiments. Data are shown as mean±SD of triplicate samples (A-F). hpi, hours post-infection; ns, not significant. *p<0.05, **p<0.01, ***p<0.001, ****p<0.0001 (two-way ANOVA).

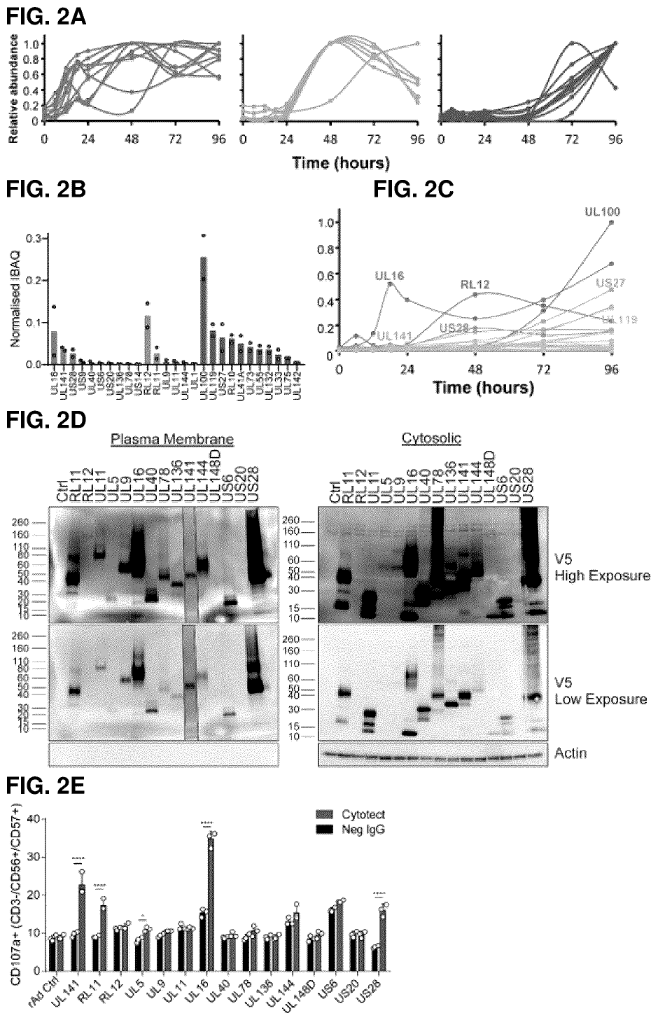

FIGS. 2A-2E: Prior work to Identify viral proteins on the plasma membrane, that could potentially prime ADCC. (A) Temporal profiles of viral proteins (n=27) identified previously on the surface of cells infected with HCMV. Proteins were only included in the analysis if detected in experiments PM1 and PM2 and quantified by ≥2 peptides in experiment PM1 or experiment PM2. Data are shown for experiment PM2. Proteins are grouped on the basis of expression kinetics, indicating that >25% of the maximal signal was reached by 24 h (left), 48 h (middle), or 72 h (right). (B) Average total abundance of each surface-expressed viral protein measured using intensity-based absolute quantification (IBAQ). Error bars indicate ranges from experiments PM1 and PM2. (C) Partitioned IBAQ abundance of each surface-expressed viral protein over time. Average IBAQ abundance values in (B) were multiplied by the fractional abundance at each time point from (A). New work to test viral proteins on the plasma membrane that can prime ADCC (D) HF-TERTs transfected with the coxsackie-adenovirus receptor (HFFF-hCARs) were transduced with RAds expressing individual viral proteins. An identical vector lacking a transgene was used as a control. Surface-expressed proteins were isolated by amino-oxybiotinylation followed by immunoprecipitation with streptavidin beads 48 h after transduction. Western blots show detection of the C-terminal V5 tags engineered into each protein, with the exception of UL141 which was detected with a UL141-specific antibody. UL141 staining of the gel was performed separately, but is overlaid on the same image. (E) Percent degranulation of CD56⁺ CD57⁺ NK cells among PBMCs in the presence of HFFF-hCARs transduced as in (D) and either Cytotect or seronegative IgGs (each at 50 μg/ml). Figure representative of three experiments. Data are shown as mean±SD of triplicate samples (E). ctrl, control. *p<0.05, ****p <0.0001 (two-way ANOVA).

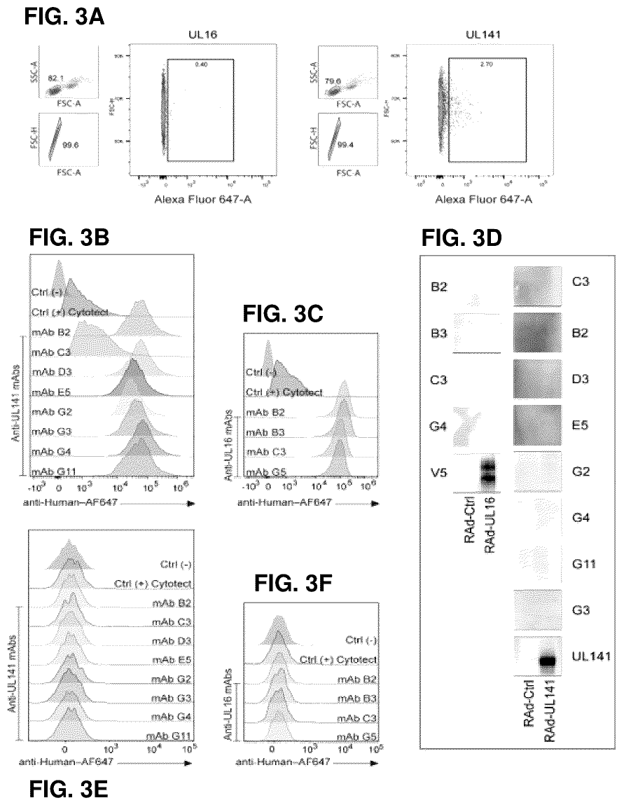

FIGS. 3A-3F: Anti-UL16 and anti-UL141 mAbs can be isolated and cloned from seropositive donors. (A) IgG+ B cells, from a HCMV seropositive donor, were stained with fluorescently labelled UL16 or UL141 proteins to sort B-cells expressing specific mAbs. (B-C) HFFF-hCARs were transduced with RAds expressing UL141 or UL16 lacking their ER retention signals. Cells were stained with the cloned human anti-UL141 or anti-UL16 mAbs and analysed by flow cytometry. Cytotect was used as a positive control. (D) HFFF-hCARs were transduced with RAd lacking a transgene, or RAds expressing wildtype forms of UL141 or UL16. Samples were lysed, separated by SDS-PAGE, and analysed by immunoblotting using the human anti-UL16 or anti-UL141 mAbs. As a positive control, the UL16 lysate was stained with an anti-V5 antibody and the UL141 lysate was stained with murine anti-UL141 antibody. (E-F) HFFF-hCARs were transduced with RAds expressing wild-type forms of UL141 or UL16. 48 h later, they were stained with human anti-UL141, anti-UL16 mAbs, or Cytotect, and analysed by flow cytometry.

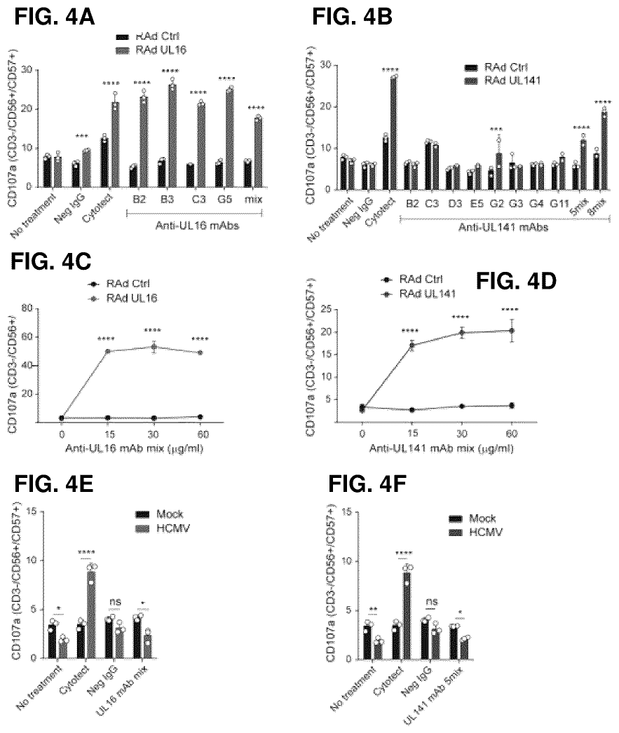

FIGS. 4A-4F: Human anti-UL16 and anti-UL141 mAbs activate ADCC efficiently against adenovirally expressed UL16 and UL141. (A-D) HFFF-hCARs were transduced with RAds expressing wildtype UL16 or UL141. An identical vector lacking a transgene was used as a control. (A) Percent degranulation of CD56⁺ CD57⁺ NK cells among PBMCs in the presence of transduced HFFF-hCARs and Cytotect (40 μg/ml), seronegative IgGs (40 μg/ml), or UL16-specific mAbs (each at 30 μg/ml). All four mAbs were included at equimolar concentrations in the mix. (B) As in (A) for UL141. Five mAbs were included at equimolar concentrations in one mix (B2, D3, G3, G4, and G11), and eight mAbs were included at equimolar concentrations in another mix (B2, C3, D3, E5, G2, G3, G4, and G11). (C) Percent degranulation of CD56⁺ CD57⁺ NK cells among PBMCs in the presence of transduced HFFF-hCARs and different concentrations of the tetravalent UL16-specific mAb mix. (D) As in (C) for the pentavalent UL141-specific mAb mix. (E, F) HF-TERTs were infected with HCMV strain Merlin. Mock-infected HF-TERTs were included as controls. (E) Percent degranulation of CD56⁺ CD57⁺ NK cells among PBMCs in the presence of infected HF-TERTs and Cytotect, seronegative IgGs, or the UL16-specific mAb mix (each at 30 μg/ml). (F) As in (E) for UL141. Experiments are representative of at least three experiments. Data are shown as mean±SD of triplicate samples (A-F). ctrl, control; ns, not significant. All experiments were performed 48 h after transduction (A-D) or infection (E, F). *p<0.05, **p<0.01, ***p<0.001, ****p<0.0001 (two-way ANOVA).

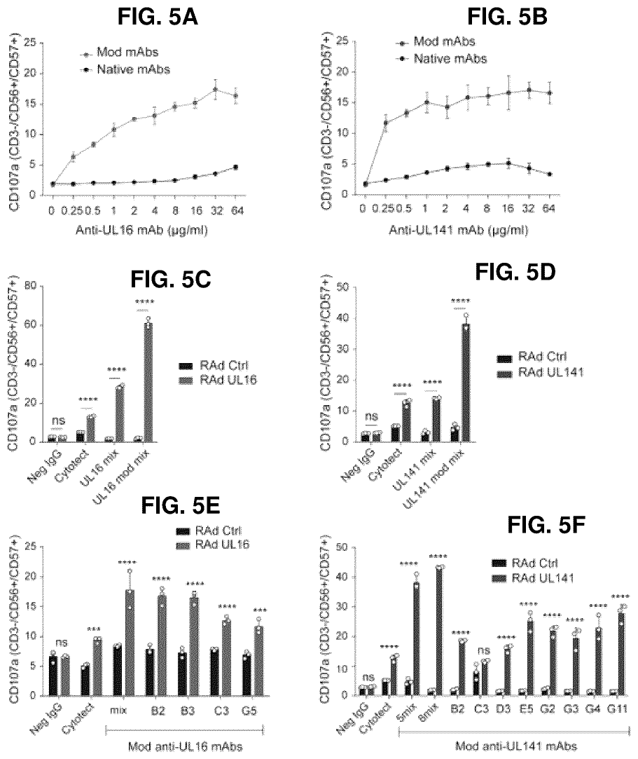

FIGS. 5A-5F: Optimised Anti-UL16 and anti-UL141 mAbs activate ADCC efficiently against adenovirally expressed UL16 and UL141. HFFF-hCARs were transduced with RAds expressing wildtype UL16 or UL141. An identical vector lacking a transgene was used as a control. (A) Percent degranulation of CD56⁺ CD57⁺ NK cells among PBMCs in the presence of transduced HFFF-hCARs and different concentrations of native or Fc-engineered (modified) UL16-specific mAbs (tetravalent mixes). (B) As in (A) for UL141 (pentavalent mixes). (C) Percent degranulation of CD56⁺ CD57⁺ NK cells among PBMCs in the presence of transduced HFFF-hCARs and Cytotect, seronegative IgGs, or tetravalent mixes of native or Fc-engineered (modified) UL16-specific mAbs (native antibodies each at 30 μg/ml, Fc-engineered (modified) mAbs each at 1 μg/ml). (D) As in (C) for UL141 (pentavalent mixes). (E) As in (C) for individual Fc-engineered (modified) UL16-specific mAbs. (F) As in (D) for individual Fc-engineered (modified) UL141-specific mAbs. Experiments are representative of at least three experiments. Data are shown as mean±SD of triplicate samples (A-F). ctrl, control; mod, modified; ns, not significant. All experiments were performed 48 h after transduction (A-F). ***p<0.001, ****p<0.0001 (two-way ANOVA).

FIGS. 6A-6H: Anti-UL141 optimised antibodies activate ADCC efficiently against HCMV. HF-TERTs were infected with HCMV strain Merlin (A-H) or Merlin ΔUL16 ΔUL141 (C, F). Mock-infected HF-TERTs were included as controls. (A) Percent degranulation of CD56⁺ CD57⁺ NK cells among PBMCs in the presence of infected HF-TERTs and different concentrations of Fc-engineered (modified) UL16-specific mAbs (tetravalent mix). (B) Percent degranulation of CD56⁺ CD57⁺ NK cells among PBMCs in the presence of infected HF-TERTs and Cytotect (40 μg/ml), seronegative IgGs (40 μg/ml), or Fc-engineered (modified) UL16-specific mAbs tested individually or in combination (each at 1 μg/ml). (C) Percent degranulation of CD56⁺ CD57⁺ NK cells among PBMCs in the presence of infected HF-TERTs and Cytotect (40 μg/ml), seronegative IgGs (40 μg/ml), or the tetravalent mix of Fc-engineered (modified) UL16-specific mAbs (each at 1 μg/ml). Activity was tested against HF-TERTs infected with Merlin or Merlin ΔUL16 ΔUL141. (D) As in (A) for UL141 (pentavalent mix). (E) As in (B) for UL141. (F) As in (C) for UL141. (G) Percent intracellular TNF-α production by CD56⁺ CD57⁺ NK cells among PBMCs in the presence of infected HF-TERTs and Cytotect (50 μg/ml), seronegative IgGs (50 μg/ml), or Fc-engineered (modified) UL141-specific mAbs tested individually or in combination (each at 1 μg/ml). (H) As in (G) for IFN-γ. Experiments are representative of at least three experiments. Data are shown as mean±SD of triplicate samples (A-H). mod, modified; ns, not significant. All experiments were performed 48 h after infection (A-F). *p<0.05, **p<0.01, ***p<0.001, ****p<0.0001 (two-way ANOVA).

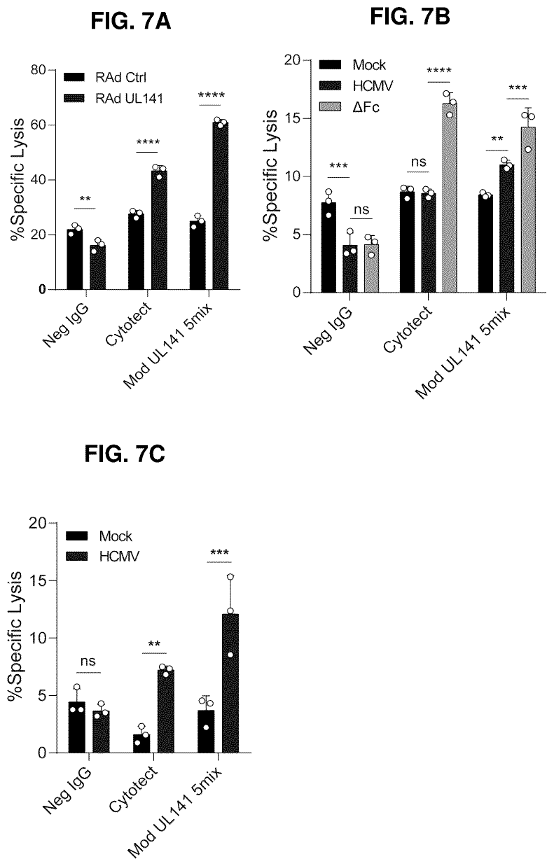

FIGS. 7A-7C: Anti-UL141 optimised antibodies mediate efficient killing of HCMV infected cells (A-C)⁵¹Cr release into the supernatant was used as a measure of the ability of NK cells to kill target cells. Targets were mixed with ex vivo purified NK cells as effectors at a E:T ratio of 20:1, then ⁵¹Cr release measured 4 h later. Seronegative IgG (50 μg/ml), Cytotect (50 μg/ml), or a mix of five Fc-engineered (modified) UL141-specific mAbs (1 μg/ml each) were included as indicated. Targets were HF-CAR infected with RAd vectors expressing UL141 (RAd-UL141), or a control vector lacking a transgene (RAd-Ctrl) (A), HFFF mock-infected, or infected with wildtype HCMV (HCMV) or HCMV lacking the viral Fc Receptors (ΔFc) (B), or ARPE19 mock infected, or infected with wildtype HCMV (C). For ARPE19 infection, cells were infected by co-culture with purified fibroblasts for 24 h, then sorted to purity. All experiments were performed 48 h after infection. Experiments are representative of at least two experiments. Data are shown as mean±SD of triplicate samples; ns, not significant, *p<0.05, **p<0.01, ***p<0.001, ****p<0.0001 (two-way ANOVA).

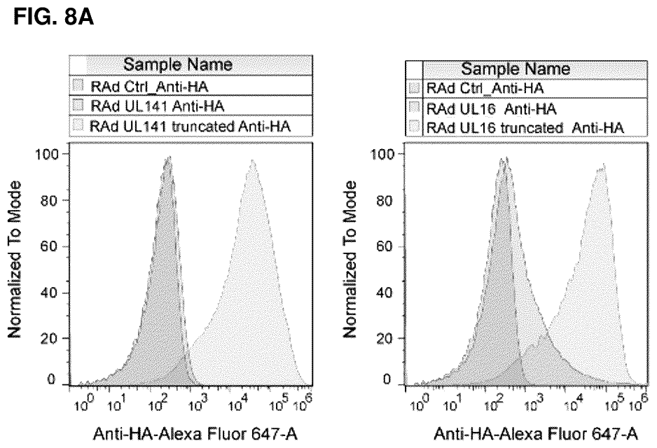

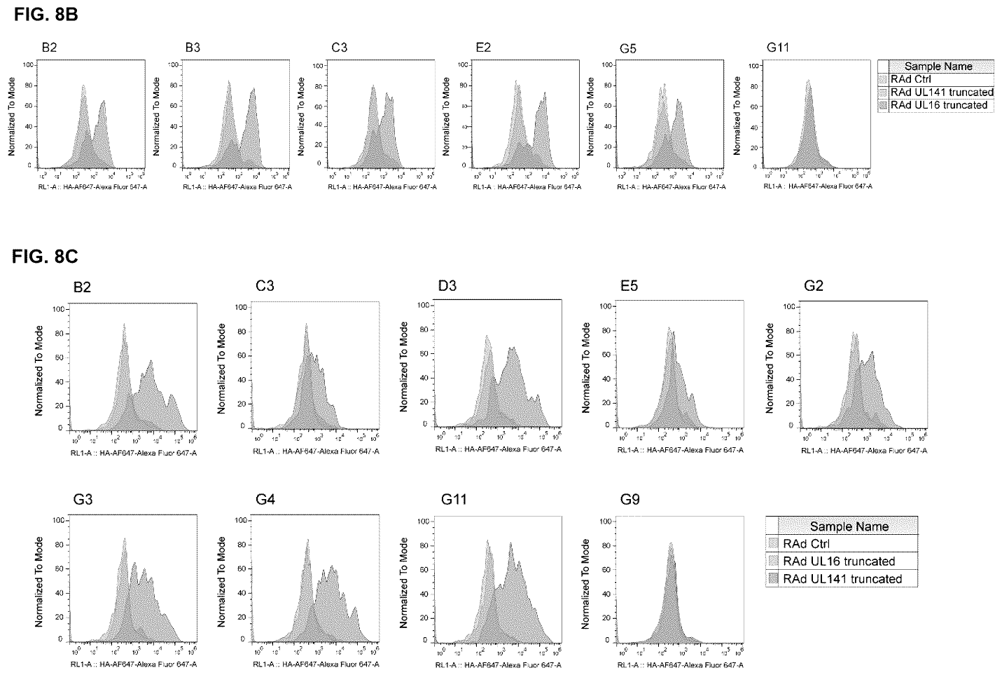

FIGS. 8A-8C: Screening of B-cell supernatants against UL16 and UL141 revealed five mAbs that bound UL16 and nine mAbs that bound UL141. (A) HFFF-hCARs were transduced with RAds expressing native or ER retention signal-truncated UL16 or UL141. An identical vector lacking a transgene was used as a control. Transduced cells were stained with a mAb specific for the HA-tag engineered into the N-terminus of each protein. (B, C) HFFF-hCARs were transduced with RAds expressing ER retention signal-truncated UL16 (B) or UL141 (C). An identical vector lacking a transgene was used as a control. Transduced cells were stained with cloned B cell supernatants and an anti-human IgG-AF647 secondary mAb. Positive clone supernatants and a representative negative clone supernatant are shown for each protein (total n=60). ctrl, control. Data are shown as flow histograms and are representative of at least two experiments (A-C).

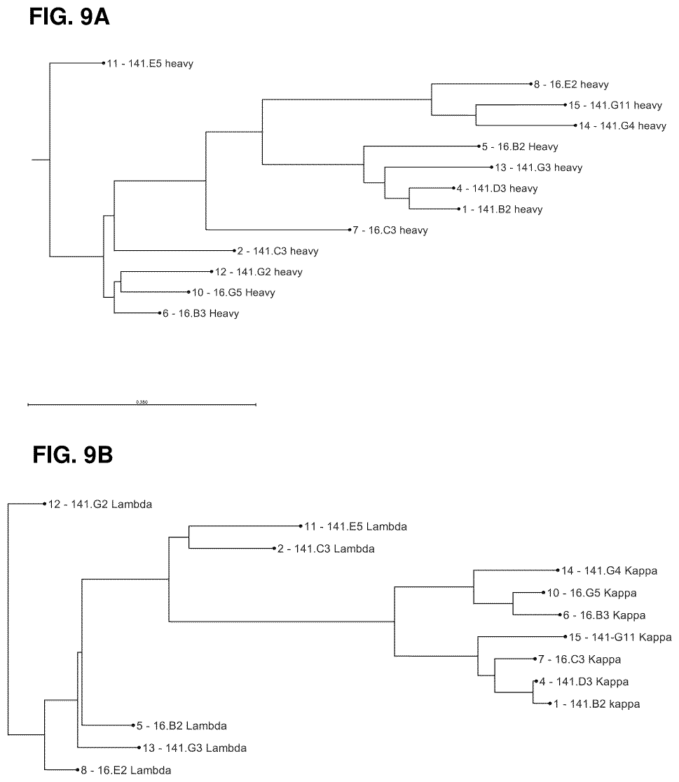

FIGS. 9A-9B: The sequences of the heavy (A) or light (B) chain of the B-cell receptor for each antibody were aligned, then a neighbour joining tree constructed using CLC Main.

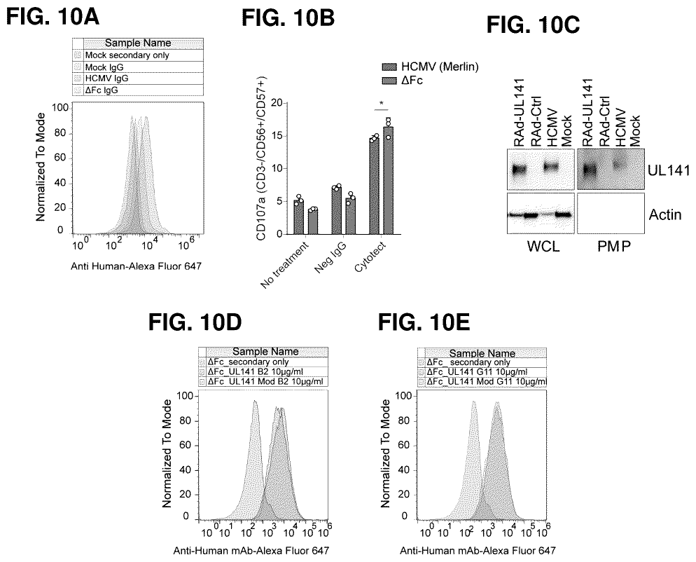

FIGS. 10A-10E: Viral Fc receptors do not have a major impact on ADCC activity at 48 hours post infection. HF-TERTs were infected with HCMV strains Merlin or Merlin ΔFc (A-C) or Merlin ΔUL141 (D, E). HF-CAR were infected with RAd expressing UL141, or control RAd lacking a transgene (C). (A) Infected cells were stained with fluorochrome-labeled Cytotect (100 μg/ml). Data are shown as flow histograms. (B) Percent degranulation of CD56⁺ CD57⁺ NK cells among PBMCs in the presence of infected cells and either Cytotect or seronegative IgGs (each at 40 μg/ml). Infected cells alone were included as a control. Data are shown as mean±SD of triplicate samples. (C) Plasma membrane proteins (PMP) were oxidised and aminoxy-biotinylated, before being immunoprecipitated with streptavidin beads, and lysed in SDS-PAGE buffer. Whole-cell lysates (WCL) prior to IP were lysed directly in SDS-PAGE buffer. Proteins were separated by SDS-PAGE, western blotted, and stained using anti-UL141 monoclonal antibodies. (D, E) Infected cells were stained with the native or Fc-modified forms of the UL141-specific mAbs B2 (C) or G11 (D), each at a concentration of 10 μg/ml, and analyzed for binding to viral FcRs. Data are shown as flow histograms. All experiments were performed 48 h after infection (A-D). *p<0.05 (two-way ANOVA).

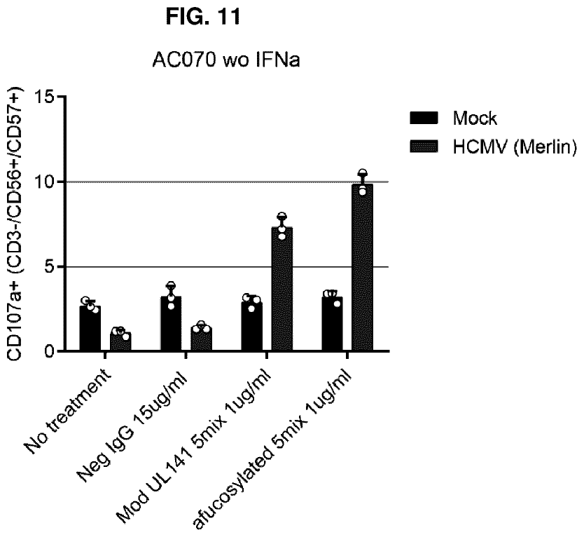

FIG. 11: Fc modified antibodies comprising Afucosylated modification activate ADCC against HCMV as efficiently as CD16 enhanced binding Fc-modified antibodies. HF-TERTs were infected with HCMV strain Merlin. Mock-infected HF-TERTs were included as controls. Percent degranulation of CD56⁺ CD57⁺ NK cells among PBMCs in the presence of infected HF-TERTs and seronegative IgGs (40 μg/ml), CD16 enhanced Fc-engineered (modified) UL141-specific mAbs tested in combination (each at 1 μg/ml), or afucosylated UL141-specific mAbs tested in combination (each at 1 μg/ml). Data are shown as mean±SD of triplicate samples (A-H). mod, (Fc) modified; ns, not significant. All experiments were performed 48 h after infection (A-F). *p<0.05, **p<0.01, ***p<0.001, ****p<0.0001 (two-way ANOVA).

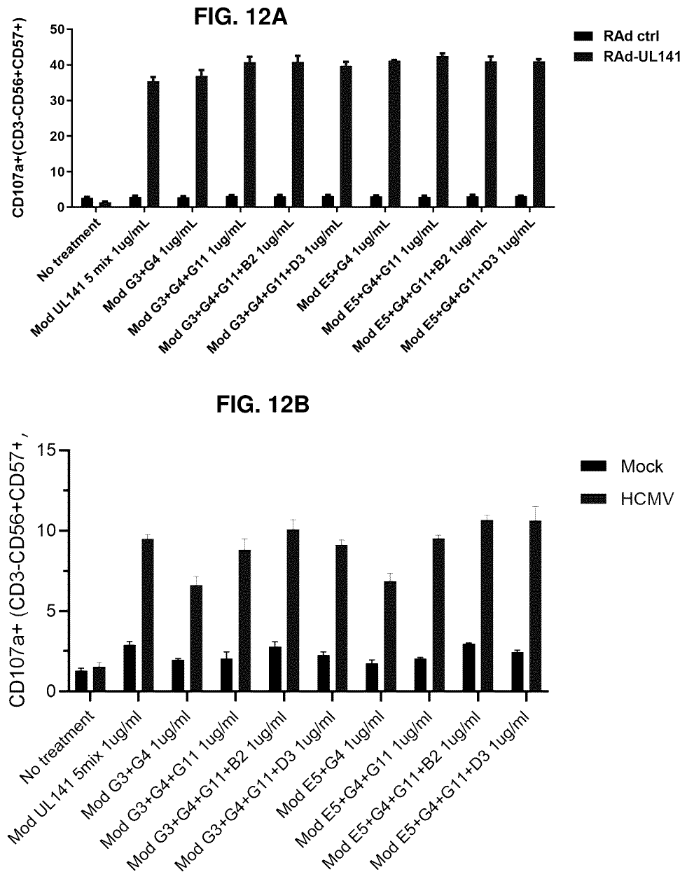

FIGS. 12A-11B: Different combinations of antibodies activate ADCC efficiently. (A) HFFF-hCARs were transduced with RAds expressing wildtype UL141. An identical vector lacking a transgene was used as a control. (B) HFFF-hTert were infected with HCMV strain Merlin, or mock infected (A-B) Percent degranulation of CD56⁺ CD57⁺ NK cells among PBMCs in the presence of these cells and the indicated combinations of anti-UL141 antibodies, used at equimolar concentrations of 1 ug/ml each. Data are shown as mean±SD of triplicate samples All experiments were performed 48 h after transduction or infection. **p<0.01, ***p<0.001, ****p<0.0001 (two-way ANOVA).

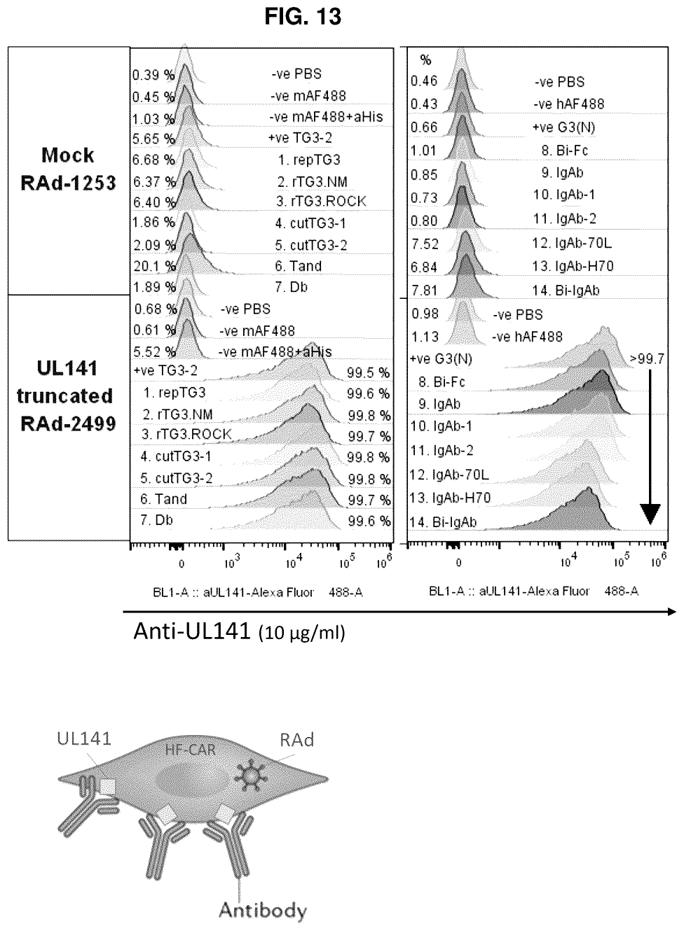

FIG. 13: anti-UL141 mAbs bind to cell-surface expressed UL141 when fused to other functional domains. HFFF-hCAR cells were infected with RAd vectors expressing UL141 (but lacking a ER-retention domain), or a control vector lacking a transgene. 48 h later, cells were dissociated and stained with the indicated antibodies, followed by a secondary antibody capable of binding to the primary antibody. For those containing a Fc domain, anti-human IgG AlexaFluor647 was used. For those lacking a Fc domain, but containing a His tag, mouse anti-his tag antibody followed by anti-mouse AlexaFluor647 was used. Stained cells were analysed by flow cytometry.

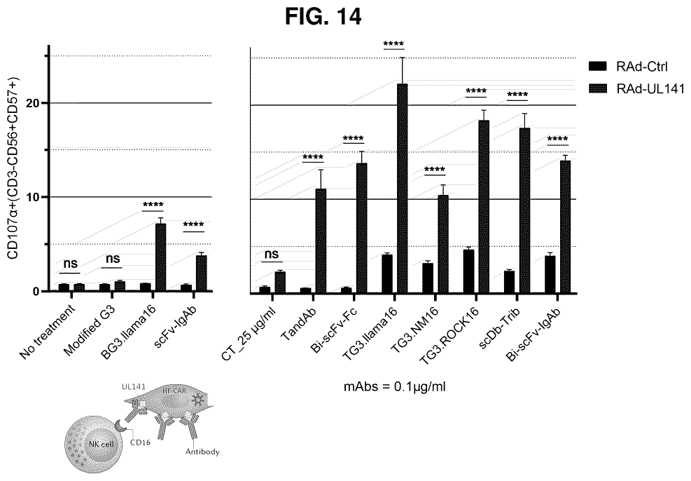

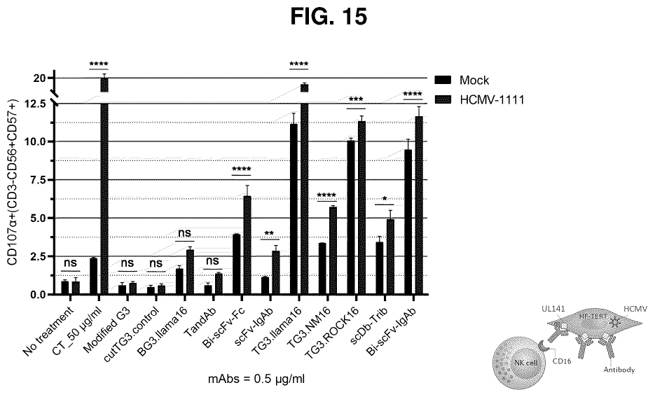

FIG. 14: anti-UL141 mAbs activate ADCC when fused to other functional domains. HFFF-hCARs were transduced with RAds expressing wildtype UL141. An identical vector lacking a transgene was used as a control. Percent degranulation of CD56⁺ CD57⁺ NK cells among PBMCs in the presence of these cells and the indicated combinations of anti-UL141 antibodies, used at 0.1 ug/ml each (with the exception of Cytotect (CT) which was used at 25 μg/ml. Data are shown as mean±SD of triplicate samples All experiments were performed 48 h after transduction or infection. *p<0.05, **p<0.01, ***p<0.001, ****p<0.0001 (two-way ANOVA) FIG. 15: anti-UL141 antibodies can activate ADCC against HCMV when fused to other functional domains. HFFF-hTert were infected with HCMV strain Merlin, or mock infected. Percent degranulation of CD56⁺ CD57⁺ NK cells among PBMCs in the presence of these cells and the indicated combinations of anti-UL141 antibodies, used at 0.5 ug/ml each. Data are shown as mean±SD of triplicate samples All experiments were performed 48 h after transduction or infection. *p<0.05, **p<0.01, ***p<0.001, ****p<0.0001 (two-way ANOVA).

### MATERIALS AND METHODS

Cells

Human fetal foreskin fibroblasts (HFFFs), HFFFs immortalized with human telomerase reverse transcriptase (HF-TERTs)(77), HF-TERTs transfected with the coxsackie-adenovirus receptor (HFFF-hCARs)(78), TERT-immortalized healthy donor skin fibroblasts (SFis) and 293 TREX cells (Thermofisher) were grown under standard conditions in Dulbecco's Modified Eagle's medium (DMEM; Thermofisher) supplemented with 10% fetal calf serum (FCS), penicillin (100 U/ml) and streptomycin (100 μg/ml). Expi293F suspension cells (Thermofisher) were maintained in a humidified shaking incubator at 150 rpm, 37° C. and 8% CO₂, and were grown in Gibco™ Expi293™ Expression Medium (Thermofisher). Ms40L low cells were a gift from Dr. Garnett Kelsoe (Duke University, USA) and Dr. David Baltimore (Caltech, USA)(79, 80). They were kept in DMEM supplemented as above with the addition of 50 μM β-mercaptoethanol.

Viruses

All viruses were derived from a bacterial artificial chromosome (BAC) containing the complete wildtype HCMV genome, with the exception of RL13 and UL128, since the absence of these genes enhances stability in fibroblasts. Mutations were engineered using either recombineering or en-passant mutagenesis, as described previously(20, 82-85). Primers sequences are listed in Table 1. Viruses were generated by transfection of BACs into HF-TERTs and titrated on HFFFs. All modifications were sequence-verified prior to BAC transfection, and all viruses were sequenced at the whole-genome level following reconstitution to exclude the occurrence of second-site mutations.

Replication-deficient Adenovirus (Rads) were generated as described previously(84). They were RAd-Ctrl (no exogenous protein-coding region), RAd-UL141AER (expressing UL141 carrying a deletion of the cytoplasmic tail and an exogenous signal peptide containing an HA tag after the cleavage site), RAd-UL16AER (expressing UL16 carrying a deletion of the cytoplasmic tail and an exogenous signal peptide containing a HA tag after the cleavage site), RAd-sUL141 (expressing the UL141 extracellular domain with a C-terminal strep tag), RAd-sUL16 (expressing the UL16 extracellular domain with a C-terminal 6His tag), RAd-UL141 (expressing the native form of UL141) and RAd-UL16 (expressing the native form of UL16). RAds expressing other HCMV proteins have been described previously, and all contained a C-terminal V5 epitope tag. All RAds were propagated by transfection of the relevant plasmids into 293 TREX cells as described previously(84).

Proteomics

Data originally published in(45) was re-analysed to estimate the absolute abundance of each cell surface viral protein. To be included in this analysis, proteins required quantitation in both experiments PM1 and PM2, by ≥2 peptides in at least one of the two experiments. Overall, this included 27/29 of the viral proteins we originally measured. Experiment PM1 examined cells infected with strain Merlin in biological duplicate at 0 h, 24 h, 48 h, and 72 h. Re-analysis was based on mean values for each time point. Experiment PM2 examined cells infected with the same HCMV strain in single replicates at 0 h, 6 h, 12 h, 18 h, 24 h, 48 h, 72 h and 96 h. In re-analysis, mean values for time point 0 were used, and infection with irradiated HCMV at 12 h was excluded from analysis. In FIG. 2A, for experiment PM2 data, proteins were grouped according to when >25% of the maximum signal was reached. Abundance for each protein was normalised to a maximum of one. For FIG. 2B, the method of intensity-based absolute quantification (IBAQ) was adapted to estimate the relative abundance of each of the 27 viral proteins. The maximum MS1 precursor intensity for each quantified peptide was determined, and a summed MS1 precursor intensity for each protein across all matching peptides was calculated, considering data for experiments PM1 and PM2 separately. Intensities were divided by the number of theoretical tryptic peptides from each protein between 7 and 30 amino acid residues in length to give estimated IBAQ values. For each of experiments PM1 and PM2, estimated IBAQ values were divided by the sum of all values to give normalised IBAQ values. The average and range of normalised IBAQ values for each protein are shown. To determine the proportion of the average normalised IBAQ values that arose at each time point of infection, IBAQ values were was adjusted in proportion to normalized TMT values shown in FIG. 2A.

Protein Purification and Labelling

Soluble UL141 and UL16 were produced in HFFF-hCARs transduced with RAd-sUL141 or RAd-sUL16, respectively, for 10 d at a multiplicity of infection (MOI) of 40 plaque-forming units (PFU)/cell. Supernatants were collected and purified using Strep-Tactin® (IBA GmbH) or HisTrap HP columns (GE Healthcare). Both proteins were subjected to buffer exchange in PBS and fluorescently labelled using the Alexa Fluor 647 Protein Labelling kit (Thermo Fischer Scientific).

Antibody Isolation

PBMCs were isolated from a healthy HCMV-seropositive donor, and IgG+ memory B cells were isolated using an IgG+ memory B-cell isolation kit (Miltenyi). The enriched B cells were stained for 30 mins at 4° C. with 2 μg/ml Alexa Fluor 647-labelled protein (soluble UL141 or UL16) and flow sorted using a BD FACSAria™ III (BD Biosciences). Single cells were sorted into individual wells containing Ms40L low feeder cells, 10% FCS, 5% human AB serum, IL4 (10 ng/ml), BAFF (10 ng/ml), IL21 (10 ng/ml) and IL2 (50 ng/ml) in a final volume of 100 μl (all cytokines from Peprotech). Cultures were supplemented with an additional 100 μl of the same medium one week later. Two weeks post coculture, 50 μl of supernatant from each of the single-cell colonies was screened by flow cytometry for binding to UL141 (RAd-UL141AER) and UL16 (RAd-UL16AER). RNA was extracted from the cells that were positive for binding using the RNEasy Plus kit (Qiagen). The antibody sequence was determined by nested RT-PCR. Sequences were analysed by IgBLAST to identify the V and J composition of the heavy and light chains, and then PCR-amplified using specific primers and cloned separately into an expression plasmid containing a human IgG1 constant domain, kindly provided by Patrick Wilson (University of Chicago, USA).

Antibody Engineering

A number of Natural Killer cell Fc enhancement modifications were undertaken to the antibodies:

CD16 Binding

S239D and I332E modifications were introduced into the Fc region of each MAb by Gibson assembly. The two fragments of the plasmid, containing overlapping regions with the desired modifications, were generated using primers GGGGGACCGGACGTCTTCCTCTTCCCCCCA (SEQ ID NO: 17) and GGTTTTCTCCTCGGGGGCTGGGAGGG (SEQ ID NO: 18), or AGGAAGACGTCCGGTCCCCCCAGGAG (SEQ ID NO: 19) and CAGCCCCCGAGGAGAAAACCATCTCCAAAGCCA (SEQ ID NO: 20). The resulting fragments were assembled using the NEBuilder HiFi DNA Assembly Cloning Kit (New England Biolabs).

Afucosylation

To produce afucosylated antibodies, Expi293 cells were transduced with a CRISPR/Cas9 plasmid targeting FUT8, then stained with FITC tagged Lens culinaris agglutinin (500 ng/ml), and cell sorted. Antibodies were then produced in this cell line in the same manner as in regular Expi293 cells.

Antibody-Like Structures (ROCK/TriKE Functional Modifications)

All the new fragments or plasmids were commercially gene synthesised (GeneArt Synthesis, Thermo Fisher Scientific). Fragments were assembled using the NEBuilder HiFi DNA Assembly Cloning Kit (New England Biolabs).

ROCK Formats:
i) Bispecific, tetravalent (bivalent for each epitope)
   scFv-IgAb 141.G3-CD16A_Heavy chain
      Signal peptide—VH 141.G3—Human IgG1 CH1-CH2-CH3 with silencing mutations—Connector (30aa)—VL CD16A—Linker (21aa)—VH CD16A
   scFv-IgAb 141.G3-CD16A Light chain
      Signal peptide—VL 141.G3 (lambda)—IgG1 CL (kappa)
ii) Tri-specific, hexavalent (bivalent for each epitope)
   scFv-IgAb 141.G3-CD16A_Heavy chain
      Signal peptide—VH 141.G3—Human IgG1 CH1-CH2-CH3 w silencing mutations—Connector (30aa)—VL CD16A—Linker (21aa)—VH CD16A
   Bi-scFv-IgAb_141.G3-4L15 Light chain
      Signal peptide—VL 141.G3 (lambda)—IgG1 CL (kappa)—Linker (SGGGG)₄SG—IL15 N72D
iii) Homodimeric, bispecific, tetravalent (bivalent for each epitope)
   Bi-scFv-Fc_141.G3-CD16A
   Signal peptide—VH 141.G3—Linker (GGGGS)₃—VL 141.G3—Human IgG1 CH2-CH3 w silencing mutations—Connector (30aa)—VL CD16A—Linker (21 aa) VH CD16A
iv) Head-to-tail homodimer, Bispecific, tetravalent (bivalent for each epitope)
   TandAb_141.G3-CD16A
   Signal peptide—VH 141.G3—Linker (GGS)₄—VL CD16A—Linker (GGS)—VH CD16A—Linker (GGS)₄—VL 141.G3—Linker (GGSG)—6His
v) Heterodimeric, bispecific, hexavalent (bivalent for each epitope) HSA: binds to human serum albumin extending the half-life.
   scDb-Trib_HSA-CD16A_Heavy chain
   Signal peptide—VH HSA—CH1—Connector (30aa)—VL CD16A—Linker (GGS)₂—VH CD16A—Linker (GGS)₇—VL CD16A—Linker (GGS)₂—VH CD16A—Linker (GGSG)—6His
   scDb-Trib_HSA-141.G3 Light chain
   Signal peptide—VL HSA—CL kappa with point-mutation at the last aa (C>S)—Connector (30aa)—VH 141.G3—Linker (GGS)₂—VL 141.G3—Linker (GGS)₇—VH 141.G3—Linker (GGS)₂—VL 141.G3

TriKE Formats:
TriKE_llamaCD16-IL15-141.G3 (short: TG3.llama16)
Signal peptide—Camelid anti-CD16—Linker (SGGGG)₄SG—IL15 N72D—Linker—VH 141.G3—Linker—VL 141.G3—Linker (GGSG)—6His
TriKE_CD16.NM3E2-IL15-141.G3 (short: TG3.NM16)
Signal peptide—NM3E2 anti-CD16—Linker (SGGGG)₄SG—IL115 N72D—Linker—VH 141.G3—Linker—VL 141.G3—Linker (GGSG)—6His
TriKE_CD16A.ROCK-IL15-141.G3 (short: TG3.ROCK16)
Signal peptide—ROCK anti-CD16A—Linker (SGGGG)₄SG—IL15 N72D—Linker—VH 141.G3—Linker—VL 141.G3—Linker (GGSG)—6His
TriKE.control.1_141.G3 (short: cutTG3.control)
Signal peptide—VH 141.G3—Linker—VL 141.G3—Linker (GGSG)—6His
TriKE.control.2.BIKE_Ilama16-141.G3 (short: BG3.llama16)
Signal peptide—Camelid anti-CD16—Linker (SGGGG)₄SG—VH 141.G3—Linker—VL 141.G3—Linker (GGSG)—6His

Sequences of the Antigen-Binding Domains

Camelid anti-CD16:
(SEQ ID NO: 31)
QVQLVESGGGLVQPGGSLRLSCAASGLTFSSYNMGWFRQAPGQGLEAVA
SITWSGRDTFYADSVKGRFTISRDNSKNTLYLQMNSLRAEDTAVYYCAA
NPWPVAAPRSGTYWGQGTLVTVSS
scFv-CD16.NM3E2:
(SEQ ID NO: 32)
EVQLVESGGGVVRPGGSLRLSCAASGFTFDDYGMSWVRQAPGKGLEWVS
GINWNGGSTGYADSVKGRFTISRDNAKNSLYLQMNSLRAEDTAVYYCAR
GRSLLFDYWGQGTLVTVSRGGGGSGGGGSGGGGSSELTQDPAVSVALGQ
TVRITCQGDSLRSYYASWYQQKPGQAPVLVIYGKNNRPSGIPDRFSGSS
SGNTASLTITGAQAEDEADYYCNSRDSSGNHVVFGGGTKLTVL
scFv-CD16A.ROCK:
(SEQ ID NO: 33)
SYVLTQPSSVSVAPGQTATISCGGHNIGSKNVHWYQQRPGQSPVLVIYQ
DNKRPSGIPERFSGSNSGNTATLTISGTQAMDEADYYCQVWDNYSVLFG
GGTKLTVLGGSGGSGGSGGSGGSGGSGGSQVQLVQSGAEVKKPGESLKV
SCKASGYTFTSYYMHWVRQAPGQGLEWMGAIEPMYGSTSYAQKFQGRVT
MTRDTSTSTVYMELSSLRSEDTAVYYCARGSAYYYDFADYWGQGTLVTV
SS
HSA:
VH HSA:
(SEQ ID NO: 37)
EVQLLESGGGLVQPGGSLRLSCAVSGIDLSNYAINWVRQAPGKGLEWIG
IIWASGTTFYATWAKGRFTISRDNSKNTVYLQMNSLRAEDTAVYYCART
VPGYSTAPYFDLWGQGTLVTVSS
VL HSA:
(SEQ ID NO: 38)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGGGTKVEIK
Cytokine domain
IL15 N72D:
(SEQ ID NO: 34)
NWVNVISDLKKIEDLIQSMHIDATLYTESDVHPSCKVTAMKCFLLELQV
ISLESGDASIHDTVENLIILANDSLSSNGNVTESGCKECEELEEKNIKE
FLQSFVHIVQMFINTS
IL15 WT:
(SEQ ID NO: 35)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

Antibody Production and Purification

Expi293F suspension cells were pelleted, resuspended at 20×10⁶ cells/ml, and transfected with the relevant light and heavy chain plasmids at a ratio of 70:30 (1.25 μg/10⁶ cells of total plasmid DNA) using polyethyleneimine (PEI) diluted in ultrapure water (3.75 μg/10⁶ cells) and 0.1% Pluronic F-68. Transfected cells were cultured for 3 h and subsequently diluted to 10⁶ cells/ml with Expi293 Expression Medium containing forskolin (10 μM). Antibody-containing supernatants were collected 7 d after transfection.

Both mAbs and antibodies from the serum of seronegative donors were purified as described previously(88). Briefly, supernatants were filtered through a 0.45 μm syringe filter and incubated overnight at 4° C. with protein G agarose beads. The following day, the bead-supernatant reactions were transferred to room temperature for 2 h and then centrifuged at 3000 rpm for 10 min. The beads were transferred to a chromatography column, washed with 5 resin-bed volumes of 1 M NaCl, and eluted twice with 2.5 resin-bed volumes of PBS. Antibodies were eluted into Tris-HCl pH 9.0 with 2.5 resin-bed volumes of glycine buffer pH 2.8 (Pierce), ensuring that the final pH was approximately 7.0. The antibodies were subsequently subjected to buffer exchange against PBS.

mAb lacking a Fc domain were engineered to contain a His-tag. For these, the Antibody-containing supernatants were purified through IMAC (immobilized metal affinity chromatography) on an AKTA™ pure liquid chromatography system (Cytiva) using a HisTrap HP column (Cytiva) and the fractions containing the protein pooled and subsequently subjected to buffer exchange against PBS.

CD107a Assays

Degranulation assays were based on the flow cytometric detection of CD107a. PBMCs were rested overnight in RPMI supplemented with 10% FCS, penicillin (100 U/ml), streptomycin (100 μg/ml), and L-glutamine (2 mM) in the absence or presence of IFN-α (1,000 U/ml). HF-TERTs (allogeneic) or SFs (autologous) were plated in DMEM without FCS and infected the following day with HCMV (MOI=5 PFU/cell). Medium was replaced at 24 h p.i. with DMEM containing 10% FCS. Assays were performed at 48 h p.i. unless stated otherwise. Targets were harvested using TrypLE Express (Gibco), preincubated for 30 min with the relevant antibody preparations, and mixed with PBMCs at an effector:target (E:T) ratio of 10:1 in the presence of GolgiStop (0.7 μl/ml, eBioscience) and anti-CD107a-PerCP-Cy5.5 (clone H4A3, BioLegend). Assays were performed in triplicate in U-bottomed 96-well plates at a final volume of 200 μl/well. Background activation was determined in wells containing effectors without targets. Cells were incubated for 5 h, washed in cold PBS, and stained with LIVE/DEAD Fixable Aqua (Thermo Fisher Scientific), anti-CD3-BV711 (clone UCHT1, BioLegend), anti-CD56-BV605 (clone 5.1H11, BioLegend), anti-CD57-APC (clone HNK-1, BioLegend), and anti-NKG2C-PE (clone 134591, R&D Systems). In some experiments, cells were also fixed/permeabilized using Cytofix/Cytoperm (BD Biosciences) and stained with anti-TNFα-BV421 (clone MAb11, BioLegend) and anti-IFNγ-PE-Cy7 (clone B27, BioLegend). Data were acquired using an AttuneNxT (Thermo Fisher Scientific) and analyzed with Attune NxT software or FlowJo software version 10 (Tree Star). All assays were repeated with multiple donors. When used directly ex vivo, NK cells from different donors can vary significantly in the magnitude of their responses, only experiments where results showed consistent patterns between donors are included. Donors included both HCMV seropositive and seronegative donors.

Chromium Release Cytotoxicity Assays

Targets were incubated with 150 Ci sodium chromate (⁵¹Cr) for 1 h, washed and allowed to leach for 1 h, then incubated with purified NK cells and antibodies. After 4 h, supernatants were removed and mixed with scintillation fluid (Optiphase HiSafe 3), before reading counts per minute (CPM) in a MicroBeta 2 (Perkin Elmer). Maximum lysis was generated using 2.5% TritonX100. Specific lysis was calculated as ‘(sample CPM−spontaneous CPM)/(Maximum CPM−spontaneous CPM).

Viral Dissemination Assays

Skin fibroblasts were infected at MOI=0.05 with a virus containing a P2A-mCherry cassette after ULi36, and a eGFP tag directly fused to UL32. 24 hours post-infection, purified ex-vivo (NK isolation kit, Miltenyi Biotec) autologous NK cells were added at a range of E:T ratios, in the presence or absence of antibody. After 8-10 days, non-adherent cells were washed off and discarded, then adherent cells were trypsinised, fixed in 4% PFA, and analysed by flow cytometry for mCherry and/or eGFP expression. To determine levels of NK-mediated control, the percentage of fluorescent cells in the presence of antibody and NK cells was normalised to the percentage of fluorescent cells in the presence of antibody alone.

Immunoblotting

HFFF-hCARs were transduced with RAd-UL141 or RAd-UL16 (MOI=5 PFU/cell) for 48 h. Whole cell lysates were collected and boiled in reducing-denaturing Nu-PAGE lysis buffer, separated by electrophoresis in Criterion TGX gels (Bio-Rad), and transferred to nitrocellulose membranes (GE Life Sciences). Membranes were blocked in TBS-T buffer with 5% dried non-fat milk and stained with either anti-V5 (Clone CV5-Pk1, Biorad) or anti-actin (A2066, Sigmaaldrich) antibodies. Proteins were visualised with SuperSignal™ West Pico PLUS chemiluminescent substrate (Thermo Scientific), and imaged on a GBOX-Chemi-XX6 gel documentation system (Syngene) operating GeneSys software.

Study Approval

Healthy adult donors provided written informed consent for the acquisition of venous blood samples and dermal fibroblasts according to the principles of the Declaration of Helsinki. Study approval was granted by the Cardiff University School of Medicine Research Ethics Committee (reference number 16/52).

Statistics

Statistical significance was determined using a 1- or 2-way ANOVA as appropriate, with Sidak post-tests. A p-value of 0.05 or less was considered as significant.

### Results

### Example 1

HCMV Infected Cells are Susceptible to ADCC During the Early Phase of Infection

We examined the ability of Cytotect (clinical-grade hyper-immune globulin (HIG) pooled from donors exhibiting high anti-HCMV neutralising titres) to enhance NK cell activation in the presence of target cells infected with a HCMV strain (Merlin) expressing the complete repertoire of virally encoded immune-evasins. Since adaptive NK cells are the primary mediators of ADCC in PBMC from HCMV seropositive donors, we examined the activation of CD56+ NK cells in the CD57+ and NKG2C+ subsets, measuring degranulation via surface mobilisation of CD107a. Both populations demonstrated a greater enhancement of degranulation when antibody was added, compared to the NKG2C−/CD57− population. However, in the majority of donors, there was a large overlap between the CD57+ and NKG2C+ populations, and the levels of degranulation were virtually indistinguishable between them. As NKG2C+NK cells are rarely present in uninfected individuals, and up to 4% of people do not harbour the corresponding gene (KLRC2), subsequent data were recorded for CD57+ NK cells.

Cytotect enhanced NK cell activation at a minimum concentration of 12.5 μg/ml and became progressively more potent as concentrations increased to 50 μg/ml, representing a relatively steep activation curve (FIG. 1A). Experiments were capped at this maximum, because increased background activation was observed with higher concentrations of immunoglobulin G antibodies (IgGs) from HCMV-seronegative donors. Interestingly, efficacy was not dependent on NK cell stimulation, since equivalent results were obtained whether or not cells were pre-incubated with IFNα (FIG. 1A-B). Given that HCMV actively represses the release of interferons, this supports an important role for ADCC in rapidly activating NK cells against HCMV without a requirement for additional stimulations.

When the sensitivity of HCMV-infected cells to ADCC was investigated over the course of infection, NK cell activation was detected as early as 24 h post infection (p.i.), irrespective of pre-incubation with IFNα, but increased dramatically at 48 h p.i. (FIG. 1C-D) before reducing slightly at 72 h p.i. This reduction may be related to the expression at this later timepoint of viral Fc receptors and other NK inhibitors, which antagonise ADCC. HCMV antigens expressed on the cell surface by 48 h p.i. are therefore recognised by naturally occurring antibodies, and act as effective targets to drive ADCC. Importantly, HCMV has a slow replication cycle, with virions not produced in significant numbers until 72 h p.i., so these observations highlighted a therapeutic opportunity to limit the dissemination of HCMV.

HCMV downregulates, but does not abrogate, the expression of endogenous human leukocyte antigen (HLA) class I molecules. NK cell activation may therefore be influenced by interactions between residual HLA-I and Killer Immunoglobulin-like Receptors (KIRs). To address this possibility, we investigated NK cell recognition of allogeneic and autologous targets in the context of ADCC. The potency of HCMV-encoded NK cell evasion functions is illustrated by the fact that uninfected autologous and allogeneic targets activated NK cells much more efficiently than the corresponding HCMV-infected targets (FIG. 1E-F). However, in both cases, the inclusion of seropositive antibody overcame the strong protective effects of HCMV-encoded NK evasion functions to stimulate high levels of NK cell activation, irrespective of preincubation with IFNα (FIG. 1E-F). Thus, the addition of anti-HCMV antibodies is able to potently activate NK cells and overcome viral immune-evasion prior to the production of new virions, irrespective of NK cell stimulation or engagement of HLA-1.

Antigens Expressed on the Cell Surface at 48 h p.i. Promote ADCC

ADCC has the potential to target infected cells during the early phase of the HCMV replication cycle. To determine which viral antigens primed ADCC, we re-analysed data from our quantitative temporal viromic investigation of the HCMV-infected cell surface proteome. There were three clear kinetic classes of protein expression (FIG. 2A). Ten proteins reached at least 25% of their maximal cell surface levels by 24 h p.i., and a further five reached at least 25% of their maximum by 48 h p.i. Thus, a substantial number of viral proteins are trafficked to the cell membrane prior to the production of new virions. Furthermore, multiple proteins reached a maximal overall abundance equal or higher to that of structural proteins expressed during the later phases of infection (FIG. 2B). Therefore, targeting proteins expressed early during the viral lifecycle is likely to be equally as effective as targeting later-expressed factors. An analysis of the partitioned abundance of each protein over time indicated that UL16, RL12, UL141 and US28 were expressed on the cell surface at 48 h p.i., were among the most abundant viral proteins at these times and would therefore be potential candidates for ADCC targets (FIG. 2C).

On the basis of these results, we generated replication-deficient adenovirus (RAd) vectors expressing each of the 15 viral proteins that were reproducibly identified on the surface of HCMV-infected cells by 48 h p.i. (FIG. 2D). Each RAd was then tested individually for its capacity to promote ADCC in the presence of pooled polyclonal HIG (FIG. 2D). UL16, UL141, US28, RL11 and UL5 each induced a significant increase in NK cell activation that was dependent on the presence of cytotect, indicating that these viral antigens could induce early-phase ADCC.

Antibodies Directing ADCC can be Isolated from Human Donors

To investigate whether the identified viral protein targets could mediate ADCC in the context of HCMV infection, we generated a series of monoclonal antibodies (mAbs). RL11 is an Fc-binding protein, which complicates both the production of specific antibodies and the analysis of functional assays. US28 is a type 3 transmembrane protein, and thus the generation of US28-specific antibodies would be less straightforward. Therefore, RL11 and US28 at present do not provide for routine target antigens. Further, since UL5 was associated with only modest levels of NK cell activation, the type 1 membrane proteins UL16 and UL141 were prioritised.

Sequences encoding the extracellular domains of each protein were cloned as modified constructs with a C-terminal 6×His-tag (UL16) or a C-terminal Strep-tag (UL141) into separate RAd vectors for expression. The corresponding proteins were purified from cell supernatants via affinity chromatography, labelled with fluorochromes, and used as probes to stain IgG+ B cells from a donor infected with HCMV. UL141-specific B cells were more numerous than UL16-specific B cells (FIG. 3A). Single antigen-specific B cells were then flow-sorted into culture medium containing CD40L⁺ feeders, interleukin (IL)-2, IL-4, IL-21, and B cell activating factor (BAFF) to generate plasma cells. All secreted mAbs were then screened against cells expressing UL16 or UL141. Both proteins contain an ER-retention signal in the C-terminal cytoplasmic domain, which restricted cell-surface expression (FIG. 8). To increase the sensitivity of this flow cytometry-based antibody screen, the cell-surface abundance of target antigens was increased by deleting this region (FIG. 8A). Screening 60 B cell supernatants against these proteins revealed that nine bound UL141 and five bound UL16 (FIG. 8B).

B cell receptor (BCR) sequencing revealed that the predicted amino acid sequences of these mAbs were diverse and incorporated both x and k light chains, suggesting that antibodies had the potential to target distinct epitopes (FIG. 9). The variable domains of these BCRs were subcloned into an expression plasmid that provided a human IgG1 backbone, with the specific purpose of optimising the utility of the antibody fusion for ADCC. When expressed, these recombinant human mAbs retained their capacity to bind to UL141 and UL16 on the cell surface (FIG. 3B-C), but not denatured antigen (FIG. 3D), suggesting that all bind to conformational epitopes.

Anti-UL16 and Anti-UL141 Human mAbs Activate ADCC when Antigen is Expressed in Isolation

Although the mAbs bound to UL16 and UL141 when optimised for high expression on the cell surface (FIG. 3B-C), binding to the natural forms was not detectable by flow cytometry (FIG. 3E-F, FIG. 8A), indicating that very low levels of these proteins naturally traffic to the cell surface. Nevertheless, ADCC assays appear more sensitive than flow cytometry, as the natural version of both genes were able to induce ADCC both with Cytotect and with mAbs (FIG. 4A-B).

Each novel UL16 mAb was readily able to drive ADCC against fibroblasts expressing wild-type UL16 with an efficiency comparable to that observed with Cytotect (FIG. 4A). The level of ADCC elicited by different anti-UL16 mAbs was remarkably similar, despite the diversity of their antigen binding (Fab) sequences. When the five mAbs were mixed together at equimolar concentrations, the ADCC effect was not enhanced beyond the level of each individual antibody. These findings suggested that each mAb targeted the same immunodominant epitope with similar efficiency, irrespective of diversity in the corresponding antigen-binding domains.

In contrast, only two of the UL141-specific mAbs were capable of mediating ADCC in isolation, and activation was weak (FIG. 4B). However, when all eight purified antibodies (B2, C3, D3, E5, G2, G3, G4, and G11) were mixed together at equal concentrations, ADCC was efficiently activated. Three of the antibodies (C3, E5, and G2) were prone to eliciting non-specific activation against control infected cells, and therefore a mixture of the other five antibodies (B2, D3, G3, G4, and G11), was tested and found to be equally capable of activating ADCC, but with reduced background levels (FIG. 4B). The fact that anti-UL141 mAbs stimulated higher levels of degranulation when used as a mixture suggests that at least some of them bind to different epitopes on UL141. In dose-titration experiments against the corresponding targets, mixtures of UL16-specific or UL141-specific mAbs maximally activated NK cells at concentrations above 15 μg/ml (FIGS. 4C & D), indicating greater efficacy compared with Cytotect (FIGS. 1A & B).

Although these results were encouraging in terms of therapeutic development, pooled mAbs specific for UL16 or UL141 were unable to activate NK cells in the presence of targets infected with HCMV, even though Cytotect was effective (FIG. 4E-F). HCMV encodes four Fc-binding proteins (FcRs; RL11, RL12, RL13 and UL119) that have the potential to antagonise ADCC. Accordingly, cells infected with an HCMV mutant strain lacking all four of these genes (HCMVΔFc) were bound by human IgGs but to a lesser extent than cells infected with wildtype HCMV (FIG. 10A). However, NK cells were activated similarly under both conditions in the presence of Cytotect (FIG. 10B). The lack of efficacy of the pools of specific antibodies against HCMV infected cells was therefore not caused by antagonism of ADCC by viral FcRs but it may reflect lower levels of protein on the cell surface during HCMV infection, compared to RAd expression (FIG. 10C), or the concerted action of multiple virally-encoded immune-evasins that inhibit NK activation. Therefore, despite showing activity in artificially expressed cells, the antibodies were not effective in eliciting a response against HCMV infected cells, reaffirming the difficulty in targeting this virus.

Antibody Engineering Enables mAbs to Activate ADCC Against HCMV

However, a major advantage of cloned mAbs is that they can be manipulated to enhance different effector functions. We took advantage of this to optimise the ability of our mAbs to activate ADCC by introducing Fc region modifications to enhance killing.

Two amino acid sequence changes into the Fc region to enhance binding to CD16 on NK cells were introduced. In line with previous data indicating that viral and host FcRs bind Fc in different ways, these modifications did not affect binding to viral FcRs (FIG. 10D-E). Dose-titration experiments revealed that mixtures of engineered mAbs specific for UL16 or UL141 activated NK cells more potently and at much lower concentrations than either the corresponding unmodified mAbs (FIGS. 5A & B) or Cytotect (FIGS. 5C & D). As before, when tested separately, all of the mAbs against UL16 activated ADCC, and there was no increase in activation when they were combined (FIG. 5E). However, unlike the unmodified versions, all the modified UL141 mAbs activated ADCC individually (FIG. 5F). Moreover, they retained the ability to show enhanced activation when used in combination, whether as a mixture of five or eight MAbs (FIG. 5F).

Next, we tested the efficiency of the mAbs in the context of HCMV infection both separately and in combination. Even in their modified form, the anti-UL16 mAbs were not able to reproducibly activate ADCC against HCMV-infected cells (FIG. 6A-C).

However, in contrast, ADCC was efficiently achieved against HCMV using the Fc CD16 binding modified anti-UL141 mAbs. Individually these mAbs only activated ADCC very weakly, but the combination of five antibodies was successful at activating ADCC almost as effectively as Cytotect, despite being used at a 40-fold lower concentration (FIG. 6D-E). This effect was highly specific, because activation was not apparent when a virus lacking the cognate antigen was used (FIG. 6F). Furthermore, these antibodies were also capable of activating NK cells to secrete TNFα and IFNγ, indicating potent antiviral effector functions in the presence of targets infected with HCMV (FIG. 6G-H).

Finally, we examined the ability of our mAbs to promote direct killing of cells. Measuring short-term cytotoxicity using chromium-release assays revealed that a mix of five modified anti-UL141 antibodies led to a substantial increase in NK-mediated cell death when UL141 was expressed in isolation (FIG. 7A), or when fibroblasts were infected with HCMV (FIG. 7B). This affect was not restricted by cell type, because similar results were obtained when HCMV infected epithelial cells were used (FIG. 7C). Furthermore, our defined antibodies significantly outperformed Cytotect in these assays, despite being used at a lower concentration. Interestingly, unlike in degranulation assays (FIG. 10B) when cytotoxicity experiments were performed the viral FcRs did limit cell death, since killing was significantly enhanced in their absence (FIG. 7B). However, this affect was more pronounced with Cytotect compared to our engineered mAbs. Thus, antibody engineering to enhance NK cell activation may also improve function by overcoming viral countermeasures. We also investigated the ability of the UL141 mAbs to promote control of virus using a recently developed 10-day viral dissemination assay (VDA), which captures the effects of both cytotoxic and non-cytotoxic virus control in a fully autologous system. The UL141 mAbs demonstrated a striking ability to enhance NK-mediated virus control in this assay, demonstrating that they can act as powerful effectors for long-term control of virus infection, even at low effector:target ratios.

Equally, efficacy of alternative Fc modifications to enhance NK cell binding was also explored through afucosylation of the Fc region. Notably, afucosylation of the antibodies was found to lead to activation of ADCC against HCMV as efficiently as CD16 Fc-modified antibodies (FIG. 11).

Through epitope mapping, minimal combinations of antibodies were investigated to determine the minimum number of antibodies, and so the minimum number of UL141 epitopes, required to be bound in order to elicit an immune reaction/cell killing in cells infected with virus, such as HCMV. As can be seen from FIG. 12, when considering those antibodies not sharing significantly overlapping epitopes, a minimum of 2 antibodies was all that was required to activate ADCC to levels comparable to those observed when testing a 5 antibody mix (FIG. 12), with maximum activity observed when using a composition of 4 antibodies. This data demonstrates that when using at least 2 antibodies binding 2 different or only partially shared epitopes on UL141, and when modified as disclosed herein, ADCC can be activated.

### Example 2

Furthermore, antibody modifications are not restricted to point mutations in the Fc domain. We developed further constructs in which the VH/VL chains were linked to a variety of enhancing modifications. They were converted into a scFv, and linked to either a scFv or nanobody capable of binding CD16, with or without a linker corresponding to the sequence of IL15. Alternatively, the VH/VL domains were either kept as separate domains, or fused into a scFv, and the Fc domain was modified to contain mutations that abrogated CD16 binding (L234F/L235E/D265A), then a scFv capable of binding to CD16 was fused to the C-terminus. Finally, the VH/VL domains were converted into scFv, and fused to a CD16-binding scFv, along with human serum albumin (HSA) binding sequences. Constructs lacking a Fc domain were engineered to contain a 6His tag for detection and purification. All formats were capable of binding to UL141 when expressed on the cell surface (FIG. 13), and all activated ADCC specifically in the presence of a cell line expressing the UL141 protein at a lower concentration than antibodies containing that those antibodies comprising a modified Fc region (FIG. 14). Furthermore, when tested against HCMV infected cells, although a single antibody (modified G3) did not activate ADCC when used at a lower concentration in isolation, these reformatted constructs led to enhanced levels of NK activation in the presence of HCMV, compared to mock infected cells (FIG. 15). Thus, they promoted ADCC against HCMV when used as antibodies in isolation and did not require multiple antibodies as per the Fc modified antibodies.

### SUMMARY

Multiple human anti-HCMV mAbs have been developed that target virus neutralisation as their mechanism of action. Although these mAbs offer advantages over hyper-immune globulin (HIG), in that they are defined products with a specific activity, the highly cell-associated nature of clinical HCMV strains and the intrinsically greater resistance to antibody neutralisation by cell-to-cell spread within a host, in comparison to cell-free entry from host to host, mean that their ability to prevent intra-host spread may be limited. In contrast, antibody-mediated activation of cellular immunity does not suffer from these limitations, and there is therefore considerable interest in exploiting this powerful mechanism of control across multiple pathogens and diseases. However, this requires that the antigens that optimally activate ADCC be mapped and cloned human mAbs capable of mediating ADCC produced. Here we demonstrate that plasma-membrane proteomics and functional immunology can be combined to identify novel ADCC targets for treatment against HCMV, a ubiquitous pathogen that causes severe disease following congenital infection and in the immunocompromised for which vaccines are licensed, and there are limited treatment options available.

As a virus that persists lifelong, HCMV faces major challenges in avoiding being cleared by the immune response, and as a result has evolved an exceptionally broad range of techniques to limit immune-activation, that means that the virus poses a particular challenge to the development of methods to activate anti-viral immunity. Here we have generated antibodies capable of reversing the ability of viral immune-evasins to inhibit NK cell activation, even when the HCMV strain expressed the complete repertoire of immune evasive genes present in a clinical isolate. As well as encoding functioning immune-evasins, it seems likely that HCMV has evolved to restrict cell-surface expression of viral proteins in order to minimise ADCC. As a result, determining surface antigen expression is no trivial task and the extreme sensitivity of mass-spectrometry was required in order to identify viral cell-surface antigens. The choice of cell-surface antigen is likely to be an important parameter that defines the efficacy of mAbs that activate ADCC and surprisingly, the antigens that we identified as mediating ADCC were not the classical viral structural proteins that ADCC studies have traditionally focused on. These targets were screened to identify the viral antigens responsible for activating ADCC, of which only antibodies targeting one of these antigens (UL141) were sufficient to mediate ADCC against HCMV infected cells, even at low concentrations. Eight UL141 antibodies were isolated, however, 3 were disregarded as they elicited non-specific activation and whilst the remaining 5 antibodies could elicit ADCC when used in combination, this was not in the context of HCMV infection.

However, an advantage of monoclonal antibodies is that they are defined products with consistent specificity over time, and molecular engineering can be used to optimise functionality for specific purposes. Accordingly, these five UL141 mAbs were genetically engineered in the Fc region and by doing so, unlike the unmodified versions, all five of the modified UL141 mAbs activated ADCC individually and in combination. Further, when in used combination and modified, their effect was comparable to the known polyclonal, cytotect, even at almost 40-fold lower concentration. In addition, the UL141 antibodies exhibit superior direct NK targeted cell killing of the virus, showing enhanced NK-mediated virus killing, demonstrating that they can act as powerful effectors for long-term control of virus infection, even at low effector:target ratios. Notably, this effect was not limited to a single type of Fc modification, but found to occur when considering various Fc modifications known to enhance NK cell effector binding.

Furthermore, in addition to Fc modified antibodies comprising the UI141 binding variable regions, further antibody constructs in which the VH/VL chains were linked to a variety of enhancing structural modifications. These antibodies were found to promote ADCC against HCMV, even when used in isolation.

Therefore, although cell surface antigen levels were extremely low, it is clear that ADCC has evolved to be extraordinarily sensitive, with antibody engineering enabling strong NK activation to occur despite antibody binding being undetectable by flow cytometry, underscoring the potential of our pipeline to produce highly effective antibodies.

The use of multiple antibodies targeting the same antigen also has the possibility to limit the selection of viral escape mutations. The sequences of UL141 are well conserved among clinical HCMV isolates, suggesting that antibodies targeting them could control a broad range of virus strains.

We have therefore identified multiple cell-surface targets for the development of novel anti-viral immunotherapies or vaccination strategies that can activate ADCC, and we have generated what we believe to be the first human antibodies targeting a single HCMV antigen that are sufficient to activate ADCC. Together these results open the path to the development of novel immunotherapeutic strategies that can activate multiple different arms of cellular immunity, enabling enhanced control of HCMV in vivo.

TABLE 1

| Primer | Sequence |
|---|---|
| UL119-F | GAGCTGGTCGCCCTGATGCAGATGCACGGTGCTGTTGGGGTTGCCGTGT |
| | GACGAGACGGCGTGTGGACGAGCTATATGTTAGGGATAACAGGGTAATG |
| | GC (SEQ ID NO: 21) |
| UL119-R | GTTTAGGCGTCACAAGAGGTGACGCGACCTCCTGCCACATATAGCTCGT |
| | CCACACGCCGTCTCGTCACACGGCAACTCAGAAGAACTCGTCAAGAAGG |
| | CG (SEQ ID NO: 22) |
| RL11- | ACGACGTCTGATAAGGAAGGCGAGAACGTCTTTTGCACCGCACTATCACA |
| 12-F | AATAATAACATGCGCAAAACAAGTCACCGTAGGGATAACAGGGTAATGGC |
| | (SEQ ID NO: 23) |
| RL11- | AGAGCCCATGTAGTGCGCGTGCCATGTGAGATGTCACGGTGACTTGTTTT |
| 12-R | GCGCATGTTATTATTTGTGATAGTGCTCAGAAGAACTCGTCAAGAAGGCG |
| | (SEQ ID NO: 24) |
| UL16-F | TGGGGTCAAAAGCCTGGGTACTTATGGGGAGCGCGCACAAAGGACCGTC |
| | AGGCGCCGGCAATAATCGAGCGCCTCTACGTAGGGATAACAGGGTAATG |
| | GC (SEQ ID NO: 25) |
| UL16-R | ATCCGGGCGGTCTCGGATATAGCGAGCCCAATCGGACGTAGAGGCGCT |
| | CGATTATTGCCGGCGCCTGACGGTCCTTTCAGAAGAACTCGTCAAGAAG |
| | GCG (SEQ ID NO: 26) |
| UL141-F | GTGAAAATACTCCAAAATCCCAAAAATGCCGCGATTCCCCGAGTGGCCCA |
| | GGGAGAGATGATTCTTTTCTTCCCTTTAGGGATAACAGGGTAATCGATTT |
| | (SEQ ID NO: 27) |
| UL141-R | CACGGAGCAGGAACAGGGGGGCAGCGTCTCTGCGAAAAAGGGAAGAAA |
| | AGAATCATCTCTCCCTGGGCCACTCGGGGGCCAGTGTTACAACCAATTAA |
| | CC (SEQ ID NO: 28) |

### REFERENCES

20. Stanton R J et al. Reconstruction of the complete human cytomegalovirus genome in a BAG reveals RL13 to be a potent inhibitor of replication. J Clin Invest 2010; 120(9):3191-3208.
45. Weekes M P et al. Quantitative temporal viromics: an approach to investigate host-pathogen interaction. Cell 2014; 157(6):1460-1472.
77. McSharry B P, Jones C J, Skinner J W, Kipling O, Wilkinson G W. Human telomerase reverse transcriptase-immortalized MRC-5 and HCA2 human fibroblasts are fully permissive for human cytomegalovirus. J. Gen. Virol. 2001; 82(Pt 4):855-863.
78. McSharry B P et al. Adenovirus E3/19K Promotes Evasion of NK Cell Recognition by Intracellular Sequestration of the NKG2D Ligands MICA and MICB. J Virol 2008; 82:4585-4594.
79. Su K Y, Watanabe A, Yeh C H, Kelsoe G, Kuraoka M. Efficient Culture of Human Naive and Memory B Cells for Use as APCs. J Immunol 2016; 197(10):4163-4176.
80. Luo X M, Maarschalk E, O'Connell R M, Wang P, Yang L, Baltimore D. Engineering human hematopoietic stem/progenitor cells to produce a broadly neutralizing anti-HIV antibody after in vitro maturation to human B lymphocytes. Blood 2009; 113(7):1422-1431.
82. Karsten Tischer B, Smith G A, Osterrieder N. En passant mutagenesis: A Two Markerless red recombination system. Methods Mol. Biol. 2010; 634:421-430.
83. Murrell I, Tomasec P, Wilkie G S, Dargan D J, Davison A J, Stanton R J. Impact of Sequence Variation in the UL128 Locus on Production of Human Cytomegalovirus in Fibroblast and Epithelial Cells. J. Virol. 2013; 87(19):10489-10500.
84. Stanton R J, McSharry B P, Armstrong M, Tomasec P, Wilkinson G W. Re-engineering adenovirus vector systems to enable high-throughput analyses of gene function. Biotechniques 2008; 45(6):659-662, 664-668.
85. Warming S, Costantino N, Court D L, Jenkins N A, Copeland N G. Simple and highly efficient BAC recombineering using galK selection. Nucleic Acids Res 2005; 33(4):e36.
88. Ho I Y et al. Refined protocol for generating monoclonal antibodies from single human and murine B cells. J Immunol Methods 2016; 438:67-70.

1. An anti-viral composition comprising at least one monoclonal antibody or a plurality of monoclonal antibodies, or at least one fragment thereof, comprising;
a plurality of different variable regions, wherein each region binds UL141 protein; and
a modified Fc region wherein the modification enhances immune cell binding or function.

2. The anti-viral composition according to claim 1 wherein said Fc modified region comprises at least one point mutation.

3. The anti-viral composition according to claim 2 wherein said Fc modified region comprises at least one point mutation at amino acid position 234, 236, 239, 243, 292, 298, 300, 305, 330, 332, 333, 334 or 396, including any combination of the afore point mutations.

4. The anti-viral composition according to claim 3 wherein said point mutation is selected from the group comprising: L234Y, G236W, G236A, S239D, F243L, R292P, S298A, Y300L, V305I, A330L, I332E, E333A, K334A, P396L, including any combination of the afore point mutations.

5. The anti-viral composition according to claim 1, wherein said Fc modified region is aglycosylated or afucosylated.

6. The anti-viral composition according to claim 1, wherein said variable region has an amino acid sequence selected from:
a)
(SEQ ID NO: 1)
DIQMTQSPSTLSASVGDRVTITCRASQSISSWLAWYQQKPGKAPKLLIY
MASSLESGVPSRFSGSGSGTEFTLTISSLQPDDFATYYCQQYNSYPRTF
GQGTKVEIK;
b)
(SEQ ID NO: 2)
QSALTQPASVSGSPGQSITISCTGTSNDVGAYNSVSWYQQHPGKAPKLM
IYDVDNRPSGVSTRFSGSKSGNTASLTISGLQPDDEADYYCSSYTSRRT
LGVFGGGTKVTVL;
c)
(SEQ ID NO: 3)
EIVLTQSPATLSLSPGERATLSCRASQSASSYVAWYQQKPGQAPRLLIY
DVSIRANGIPARFSGSGSGTDFALTISSLEPEDFALYYCQHRNNWGSTF
GQGTRLEIK;
d)
(SEQ ID NO: 4)
DIQMTQSPSTLSASVGDRVTITCRASQSISKWVAWYQLKSGKVPKLLIY
QASDLQSGVPTRFSGSGSGTEFTLTIRGLQSDDFATYYCQQFDHSPWTF
GQGTKVEIK;
e)
(SEQ ID NO: 5)
DIQMTQSPSTLSASVGDRVTITCRASQSVSGWLAWYQQKPGKAPKLLIY
MASSLEGGVPSRFSGSGSGTEFTLTISSLQPDDFATYYCQQYNSYPRTF
GQGTKVEIK;
f)
(SEQ ID NO: 6)
QSVLTQPPSVSAAPGQKVTISCSGSSSNIGNNYVSWYQQLPGTAPKLLI
YDNNKRPSGIPDRFSGSKSGTSATLGITGLQTGDEADYYCGTWDSSLLE
VVFGGGTKLTVL;
g)
(SEQ ID NO: 7)
QSVLTQPPSASGTPGQRVTISCSGGSSNIGSNPVNWYQQIPGTAPKLLI
YSDDQRPSGVPDRFSGSKSGSSASLAIRGLQSEDEADYFCAARDDSLNG
PIFGGGTKLTVL;
h)
(SEQ ID NO: 8)
QSALTQPASVSGSPGQSITISCIGTSSDVGKNNLVSWYQQYPDKAPKLM
IYDVTKRPSGVSNRFSGSKSGNMASLTISGLQTEDEAHYYCCSYAGVGG
HILWVFGGGTKVTVL;
and/or
i) a variable region that shares at least 85% identity with any one of variable regions a)-g) (i.e. SEQ ID NO: 1-8);
j)
(SEQ ID NO: 9)
EVQLVESGGDLVQPGGSLRLSCAASGFIVSSNYMSWVRQAPGKGLEWVS
VIHSDGPTFYADSVKGRFTISRDSSKNMLYLQMNSLRAEDTAVYYCTRG
EFASGLYGSAGSNAFDFWGQGTLVTVSS;
k)
(SEQ ID NO: 10)
EVQLVESGGGLVQPGGSLRLSCVASTFTISPYWMSWVRQAPGKGLEWVA
NIKDDGSERYYVDSVKGRFTISRDNAKNSVFLQMNSLRAEDTATYYCAR
PGPDAFSTGWSNWFDPWGQGMLVTVSS;
l)
(SEQ ID NO: 11)
QVQLQESGPGLVRPSQTLSLTCTVSGASITSGSYYWTWIRQPAGEGLEW
LGRINTRGNINYKPSLRSRLTFSVDTSKNQFSLQLSSVTAADSAVYFCA
RVGLYDTYYYFMDVWGKGTTVTVSS;
m)
(SEQ ID NO: 12)
QVQLQESGPGLVRPSETLSLTCTVSGASVSAYYWTWIRHSPGRGLEWIG
DIYFNGKFNYNPSLESRVTISRGPSKTQLSLKLSSVTAADSAVYYCARI
GDSTMAPLYYFYYIDVWGKGTTVTVSS;
n)
(SEQ ID NO: 13)
EVQLVESGGGLVQPGGSLRLSCAASAFTVSSMYMNWVRQAPGKGLEWV
SVIYSDGTTYYRDSVKGRFTISRDNSKNKVYLQMNSLRAEDTAVYYCAR
GEFASGWYGSAGSNAFDIWGRGTMVTVSS;
o)
(SEQ ID NO: 14)
EVQLVQSGAEVKKPGASVKVSCKASGYTFTNYAISWVRQAPGQGLEWMG
WISAYNGNTNYAQKLQGRVTMTTDTSTSTAYMELRSLRSDDTAVYYCAR
VGTMVRGVIYNKRPYYYYYMDVWGKGTTVTVSS;
p)
(SEQ ID NO: 15)
EVQLVQSGAEVRKPGSSVKLSCKASGGTFRNYAMSWMRQAPGQGFEWV
GGIVPFLGKTNYAQKFQGRVTISTDESTSTAYMELSRLTSDDTAVYFCA
RGPPPVMVRGIHRTGGDWFDPWGQGTLVTVSS;
q)
(SEQ ID NO: 16)
EVQLVQSGAELKKPGSSVKVSCKASGGTFSFHAINWVRQAPGQGLEWMG
GIIPVSDTTNYAQKFHSRLTITADESTSTSYMQLTSLTDEDTAVYYCAR
EYGPVATGFDPWGQGTLVTVSS;)
and/or
r) a variable region that shares at least 85% identity with any one of variable regions j)-q).

7. The anti-viral composition according to claim 6 wherein said variable region whose amino acid sequence is selected from the group comprising or consisting of sequences a)-i) is a light chain variable region.

8. The anti-viral composition according to claim 6 wherein said variable region whose amino acid sequence is selected from the group comprising or consisting of sequences j)-r) is a heavy chain variable region.

9. The anti-viral composition according to claim 1, wherein said monoclonal antibody, plurality of monoclonal antibodies, or said at least one fragment thereof, comprise at least one heavy and at least one light chain variable region.

10. The anti-viral composition according to claim 6, wherein said monoclonal antibody plurality of monoclonal antibodies, or said at least one fragment thereof, comprise:
at least one light chain variable region selected from the group comprising or consisting of a)-i) and at least one heavy chain variable region selected from the group comprising or consisting of j)-r), including any combination thereof;
at least one light chain variable region(s) selected from the group comprising or consisting of a)-e), g) and i) and at least one heavy chain variable region(s) selected from the group comprising or consisting of j)-n), p) and r), including any combination thereof; or
at least one pair of a light and heavy chain variable region selected from the pairs in the group comprising or consisting of:
i) variable region a) and j);
ii) variable region b) and k);
iii) variable region c) and l);
iv) variable region d) and m);
v) variable region e) and n);
vi) variable region f) and o);
vii) variable region g) and p);
viii) variable region h) and q); and/or
ix) two variable regions, each one having at least 85% identity with one variable region selected from the group comprising a)-h) and j)-q).

11.-13. (canceled)

14. The anti-viral composition according to claim 1, wherein said Fc region is an alpha, mu, gamma, epsilon, or delta isotype Fc region, or a fusion product thereof.

15. The anti-viral composition according to claim 1, wherein said Fc region comprises at least one Fc modification that increases serum half-life.

16. The anti-viral composition according to claim 15, wherein said Fc modification comprises
at least point mutation at an amino acid position selected from the group comprising or consisting of 250, 252, 254, 256 and 428, including any combination of the afore point mutations; or
at least point mutation at an amino acid position selected from the group comprising or consisting of T250Q, M252Y, S254T, T256E and M428L, including any combination of the afore point mutations.

17. (canceled)

18. The anti-viral composition according to claim 1, wherein said at least one fragment comprises at least one variable region including at least one Complementarity Determining Region (CDR) for UL141 and an Fc region.

19. The anti-viral composition according to claim 18 wherein said at least one fragment comprises a plurality of different variable regions including and a plurality of Complementarity Determining Regions (CDRs) for UL141 and an Fc region.

20. An immunogenic agent or vaccine comprising the anti-viral composition according to claim 1 and a pharmaceutically acceptable excipient or carrier.

21. A pharmaceutical composition comprising the anti-viral composition according to claim 1 and a pharmaceutically acceptable excipient or carrier.

22. A combination therapeutic comprising the anti-viral composition according to claim 1 and at least one other therapeutic agent.

23.-25. (canceled)

26. A method of treating a viral infection, comprising administering said anti-viral composition of claim 1 to an individual having, or suspected of having, a viral infection.

27. The method according to claim 26 wherein said anti-viral composition is administered within 72 hour of infection or likely infection or after exposure to said virus.

28. A method of vaccinating against a viral infection comprising administering said immunogenic agent or vaccine according to claim 20 to an individual.

29. The method according to claim 26, wherein said infection is a HCMV infection.


SEQUENCE LISTING (38 sequences)

SEQ ID NO: 1 - protein, 107 aa, Artificial Sequence
  antibody variable region D3
DIQMTQSPSTLSASVGDRVTITCRASQSISSWLAWYQQKPGKAPKLLIYMASSLESGVPSRFSGSGSGTEFTLTISSLQPDDFATYYCQQYNSYPRTFGQGTKVEIK

SEQ ID NO: 2 - protein, 111 aa, Artificial Sequence
  UL141 variable region G3
QSALTQPASVSGSPGQSITISCTGTSNDVGAYNSVSWYQQHPGKAPKLMIYDVDNRPSGVSTRFSGSKSGNTASLTISGLQPDDEADYYCSSYTSRRTLGVFGGGTKVTVL

SEQ ID NO: 3 - protein, 107 aa, Artificial Sequence
  UL141 variable region G4
EIVLTQSPATLSLSPGERATLSCRASQSASSYVAWYQQKPGQAPRLLIYDVSIRANGIPARFSGSGSGTDFALTISSLEPEDFALYYCQHRNNWGSTFGQGTRLEIK

SEQ ID NO: 4 - protein, 107 aa, Artificial Sequence
  UL141 variable region G11
DIQMTQSPSTLSASVGDRVTITCRASQSISKWVAWYQLKSGKVPKLLIYQASDLQSGVPTRFSGSGSGTEFTLTIRGLQSDDFATYYCQQFDHSPWTFGQGTKVEIK

SEQ ID NO: 5 - protein, 107 aa, Artificial Sequence
  UL141 variable region B2
DIQMTQSPSTLSASVGDRVTITCRASQSVSGWLAWYQQKPGKAPKLLIYMASSLEGGVPSRFSGSGSGTEFTLTISSLQPDDFATYYCQQYNSYPRTFGQGTKVEIK

SEQ ID NO: 6 - protein, 110 aa, Artificial Sequence
  UL141 variable region C3
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLLEVVFGGGTKLTVL

SEQ ID NO: 7 - protein, 110 aa, Artificial Sequence
  UL141 variable region E5
QSVLTQPPSASGTPGQRVTISCSGGSSNIGSNPVNWYQQIPGTAPKLLIYSDDQRPSGVPDRFSGSKSGSSASLAIRGLQSEDEADYFCAARDDSLNGPIFGGGTKLTVL

SEQ ID NO: 8 - protein, 113 aa, Artificial Sequence
  UL141 variable region G2
QSALTQPASVSGSPGQSITISCIGTSSDVGKNNLVSWYQQYPDKAPKLMIYDVTKRPSGVSNRFSGSKSGNMASLTISGLQTEDEAHYYCCSYAGVGGHILWVFGGGTKVTVL

SEQ ID NO: 9 - protein, 126 aa, Artificial Sequence
  UL141 variable region D3
EVQLVESGGDLVQPGGSLRLSCAASGFIVSSNYMSWVRQAPGKGLEWVSVIHSDGPTFYADSVKGRFTISRDSSKNMLYLQMNSLRAEDTAVYYCTRGEFASGLYGSAGSNAFDFWGQGTLVTVSS

SEQ ID NO: 10 - protein, 125 aa, Artificial Sequence
  UL141 variable region G3
EVQLVESGGGLVQPGGSLRLSCVASTFTISPYWMSWVRQAPGKGLEWVANIKDDGSERYYVDSVKGRFTISRDNAKNSVFLQMNSLRAEDTATYYCARPGPDAFSTGWSNWFDPWGQGMLVTVSS

SEQ ID NO: 11 - protein, 123 aa, Artificial Sequence
  UL141 variable region G4
QVQLQESGPGLVRPSQTLSLTCTVSGASITSGSYYWTWIRQPAGEGLEWLGRINTRGNINYKPSLRSRLTFSVDTSKNQFSLQLSSVTAADSAVYFCARVGLYDTYYYFMDVWGKGTTVTVSS

SEQ ID NO: 12 - protein, 125 aa, Artificial Sequence
  UL141 variable region G11
QVQLQESGPGLVRPSETLSLTCTVSGASVSAYYWTWIRHSPGRGLEWIGDIYFNGKFNYNPSLESRVTISRGPSKTQLSLKLSSVTAADSAVYYCARIGDSTMAPLYYFYYIDVWGKGTTVTVSS

SEQ ID NO: 13 - protein, 126 aa, Artificial Sequence
  UL141 variable region B2
EVQLVESGGGLVQPGGSLRLSCAASAFTVSSMYMNWVRQAPGKGLEWVSVIYSDGTTYYRDSVKGRFTISRDNSKNKVYLQMNSLRAEDTAVYYCARGEFASGWYGSAGSNAFDIWGRGTMVTVSS

SEQ ID NO: 14 - protein, 131 aa, Artificial Sequence
  UL141 variable region C3
EVQLVQSGAEVKKPGASVKVSCKASGYTFTNYAISWVRQAPGQGLEWMGWISAYNGNTNYAQKLQGRVTMTTDTSTSTAYMELRSLRSDDTAVYYCARVGTMVRGVIYNKRPYYYYYMDVWGKGTTVTVSS

SEQ ID NO: 15 - protein, 129 aa, Artificial Sequence
  UL141 variable region E5
EVQLVQSGAEVRKPGSSVKLSCKASGGTFRNYAMSWMRQAPGQGFEWVGGIVPFLGKTNYAQKFQGRVTISTDESTSTAYMELSRLTSDDTAVYFCARGPPPVMVRGIHRTGGDWFDPWGQGTLVTVSS

SEQ ID NO: 16 - protein, 120 aa, Artificial Sequence
  UL141 variable region G2
EVQLVQSGAELKKPGSSVKVSCKASGGTFSFHAINWVRQAPGQGLEWMGGIIPVSDTTNYAQKFHSRLTITADESTSTSYMQLTSLTDEDTAVYYCAREYGPVATGFDPWGQGTLVTVSS

SEQ ID NO: 17 - DNA, 30 bp, Artificial Sequence
  CD16 primer
gggggaccggacgtcttcctcttcccccca

SEQ ID NO: 18 - DNA, 26 bp, Artificial Sequence
  CD16 primer
ggttttctcctcgggggctgggaggg

SEQ ID NO: 19 - protein, 26 aa, Artificial Sequence
  CD16 primer
AGGAAGACGTCCGGTCCCCCCAGGAG

SEQ ID NO: 20 - DNA, 33 bp, Artificial Sequence
  CD16 primer
cagcccccgaggagaaaaccatctccaaagcca

SEQ ID NO: 21 - DNA, 100 bp, Artificial Sequence
  UL119 F primer
gagctggtcgccctgatgcagatgcacggtgctgttggggttgccgtgtgacgagacggcgtgtggacgagctatatgttagggataacagggtaatggc

SEQ ID NO: 22 - DNA, 100 bp, Artificial Sequence
  UL119 R primer
gtttaggcgtcacaagaggtgacgcgacctcctgccacatatagctcgtccacacgccgtctcgtcacacggcaactcagaagaactcgtcaagaaggcg

SEQ ID NO: 23 - DNA, 100 bp, Artificial Sequence
  RL11-12-F primer
acgacgtctgataaggaaggcgagaacgtcttttgcaccgcactatcacaaataataacatgcgcaaaacaagtcaccgtagggataacagggtaatggc

SEQ ID NO: 24 - DNA, 100 bp, Artificial Sequence
  RL11-12-R primer
agagcccatgtagtgcgcgtgccatgtgagatgtcacggtgacttgttttgcgcatgttattatttgtgatagtgctcagaagaactcgtcaagaaggcg

SEQ ID NO: 25 - DNA, 100 bp, Artificial Sequence
  UL16 F primer
tggggtcaaaagcctgggtacttatggggagcgcgcacaaaggaccgtcaggcgccggcaataatcgagcgcctctacgtagggataacagggtaatggc

SEQ ID NO: 26 - DNA, 100 bp, Artificial Sequence
  UL16 R Primer
atccgggcggtctcggatatagcgagcccaatcggacgtagaggcgctcgattattgccggcgcctgacggtcctttcagaagaactcgtcaagaaggcg

SEQ ID NO: 27 - DNA, 100 bp, Artificial Sequence
  UL141 F Primer
gtgaaaatactccaaaatcccaaaaatgccgcgattccccgagtggcccagggagagatgattcttttcttccctttagggataacagggtaatcgattt

SEQ ID NO: 28 - DNA, 100 bp, Artificial Sequence
  UL141 R Primer
cacggagcaggaacaggcgggcagcgtctctgcgaaaaagggaagaaaagaatcatctctccctgggccactcgggggccagtgttacaaccaattaacc

SEQ ID NO: 29 - protein, 338 aa, Human cytomegalovirus
MCRRESLRTLPWLFWVLLSCPRLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRRLPTYDSYPGVKKMKR

SEQ ID NO: 30 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 31 - protein, 122 aa, Artificial Sequence
  CD16 variable region
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 32 - protein, 239 aa, Artificial Sequence
  CD16 Variable region
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 33 - protein, 247 aa, Artificial Sequence
  CD16 variable region
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGSGGSGGSGGSGGSGGSGGSQVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 34 - protein, 114 aa, Homo sapiens
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 35 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 36 - protein, 609 aa, Homo sapiens [sequence omitted]

SEQ ID NO: 37 - protein, 121 aa, Artificial Sequence
  HSA variable region
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 38 - protein, 110 aa, Artificial Sequence
  HSA variable region
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK